(19) United States
(12) Patent Application Publication
(10) Pub. No.: US 2009/0143426 A1
(43) Pub. Date: Jun. 4, 2009

(54) Synthesis of 1,3,6-trisubstituted-2-carboxyquinol-4-ones as selective ET A antagonists and their use as medicaments

(76) Inventors: Ralph Anthony Stephani, Mamaroneck, NY (US); Hardik Jitendra Patel, Flushing, NY (US); Sandra Eve Reznik, Larchmont, NY (US); Jerome Owen Cantor, Brooklyn, NY (US)

Correspondence Address: Ralph A. Stephani, 337 Florence Street, Mamaroneck, NY, 10543, US

(21) Appl. No.: 12/283,215

(22) Filed: Sep. 10, 2008

Related U.S. Application Data
(60) Provisional application No. 60/992,255, filed on Dec. 4, 2007.

(51) Int. Cl. A61K 31/4709; A61P 9/00; A61P 11/00; A61P 1/00; A61P 7/00

(52) U.S. Cl. 514/312

(57) ABSTRACT

The invention discloses the composition and preparation of various 1,3,6-trisubstituted-2-carboxy-quinol-4-ones of the formula 1 where R is H, alkyl, haloalkyl or hydroxyalkyl, R′ is alkyl, nitro, halogen or NR₂′″ where R′″ is alkyl or cycloalkyl, and R″ is H or alkyl. The composition of the invented compounds as methods of antagonizing the action of endothelin-1 to treat cardiovascular, pulmonary diseases and obstetric disorders and preterm labor and preeclampsia in mammals is disclosed.

### RELATED APPLICATIONS

This application claims benefit of provisional application No. 60/993,255 filed on Sep. 11, 2007.

### SUMMARY OF THE INVENTION

The subject invention is directed to the synthesis of a novel series of 1,3,6-trisubstituted-2-carboxy-quinol-4-ones having the formula 1 for the purpose of antagonizing the action of endothelin-1. A pharmaceutically acceptable amount of one or more of the novel compounds can be used for the treatment of cardiovascular, respiratory, vascular, obstetrical, oncologic or pain-related disorders. The endothelin receptor antagonist may be administered alone, or with a carrier such as saline solution, DMSO, an alcohol, or water. Administration of the endothelin-A receptor antagonist (ET_{A}-RA) may be by oral, intravenous, intraperitoneal, intramuscular, subcutaneous, sublingual, intravaginal or rectal administration or may be performed by aerosol, which can be generated by a nebulizer, or by instillation. The treatment is intended for a wide variety of human subjects, ranging from neonates to elderly subjects.

### BRIEF DESCRIPTION OF FIGURES

FIG. 1. The formula (1) of the 1,3,6-trisubstituted-2-carboxy-quinol-4-ones.

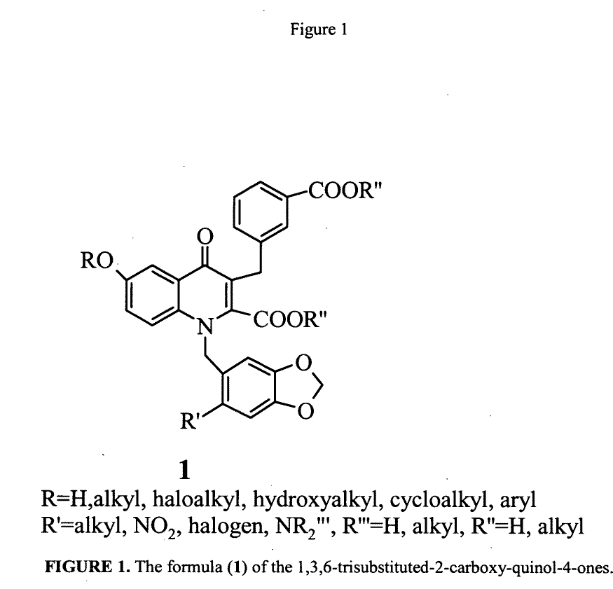

FIG. 2. The prototype compound (HJP272) inhibits specific ligand, [¹²⁵I] ET-1 (224 pM) binding to ET_{A} receptor. IC50=70.1 nM.

FIG. 3. Animals treated with LPS followed by HJP272 had significantly better control of premature delivery than animals treated with LPS followed by vehicle. (P<0.0001.)

FIG. 4. Animals treated with LPS followed by HJP272 had significantly greater prevention of premature expulsion of pups than animals treated with LPS followed by vehicle. (P<0.0001.)

FIG. 5. Animals receiving ET-1 prior to cigarette smoke exposure had a significantly higher percentage of bronchoalveolar lavage fluid neutrophils than controls treated only with cigarette smoke. The effect of ET-1 was reversed by pretreatment with HJP272.

### DETAILED DESCRIPTION OF THE INVENTION

The subject invention is directed toward the treatment of a variety of diseases in a mammal by administering one or more endothelin-1 antagonists that comprise a pharmaceutically acceptable amount of any one or combination of two or more of a novel series of 1,3,6-trisubstituted-2-carboxy-quinol-4-ones and pharmaceutically acceptable carrier and a method of antagonizing the action of endothelin-1, consisting of administering to a human a pharmaceutically effective amount of any one or combination of two or more of a novel series of 1,3,6-trisubstituted-2-carboxy-quinol-4-ones.

The diseases that may be treated with the aforementioned compounds include hypertension, congestive heart failure, restenosis following arterial injury, reperfusion injury, angina, acute or chronic pulmonary hypertension, cerebral ischemia, myocardial ischemia, cerebral vasospasm, atherosclerosis, emphysema, asthma, bronchitis, bronchiectasis, pneumonia, adult respiratory distress syndrome, neonatal respiratory distress syndrome, bronchopulmonary dysplasia, interstitial pulmonary fibrosis, cystic fibrosis, persistent pulmonary hypertension of the newborn, proliferative diseases and neoplasia, especially prostate cancer, acute and chronic renal failure, cyclosporine-induced nephrotoxicity, gastric ulceration, colitis, inflammatory bowel disease, migraine, Raynaud's disease, erectile dysfunction, endotoxin-induced toxicity, LPL-related lipoprotein disorders, platelet aggregation, thrombosis, IL-2 mediated cardiotoxicity, preterm labor, premature rupture of membranes, placental abruption, pre-eclampsia, miscarriage, stillbirth, pain, especially cancer-related bone pain, infertility, malaria, sleeping sickness, Chagas disease, cerebral malaria and leukomalacia. The endothelin-1 antagonist may be administered orally, intravenously, topically, intramuscularly, intratracheally, or by any other route deemed efficacious.

Typically, the effective daily amount of endothelin antagonist is from about 1 μg/kg to 10 mg/kg of body weight. It may be given once per day or more often, until the desired daily dosage is fully administered. The endothelin receptor antagonist (ET_{A}-RA) is administered in a pharmaceutically acceptable carrier. Such examples include saline solution, DMSO, an alcohol solution, sodium carbonate solution, or water. Such carriers are well known in the art, and the specific carriers employed may be varied depending upon factors such as size of the subject being treated, treatment dose, and the like. Further, the time over which the endothelin receptor antagonist (ET_{A}-RA) is administered may vary as is well known in the art to achieve the desired results. The treatment is intended for a wide variety of human subjects, ranging from neonates to elderly subjects.

### BACKGROUND OF THE INVENTION

### Introduction

Endothelin-1, an extremely potent vasoconstrictor peptide (Yanagisawa et al. 1988) has now been implicated in a broad spectrum of physiologic and pathologic processes, including cardiovascular, respiratory, vascular, obstetrical, oncologic or pain-related disorders. For example, the peptide increases myometrial smooth muscle tone (Kaya et al. 1999, Yallampali and Garfield 1994, Wollf et al. 1993) and it has also been shown that infection and inflammatory cytokines stimulate ET-1 production (Woods et al. 1999).

We have found that treatment of pregnant mice with lipopolysaccharide results in increased levels of expression of both ET-1 and ECE-1 (Wang et al 2008). We have also shown that treating LPS-stimulated pregnant mice with the ECE-1 inhibitor phosphoramidon decreases the incidence of premature delivery (Koscica et al. 2004).

In addition, we have recently found that treating LPS-stimulated pregnant mice with the selective ET_{A}-RA BQ-123 also results in decreased incidence of premature delivery and that this effect is dose dependent (Wang et al. 2008).

Finally, we have also recently found that RNAi directed at endothelin-converting enzyme-1 results in a dramatic decrease in the incidence of premature delivery (Wang et al. 2008).

In a separate line of investigation, we have shown that inhibition of ET-1 activity with either phosphoramidon or an endothelin receptor antagonist markedly reduces lipopolysaccharide (LPS)-induced influx of neutrophils into the lung (Bhavsar et. al. 2008a, Bhavsar et al. 2008b). Measurement of bronchoalveolar lavage fluid (BALF) leukocytes demonstrated a selective recruitment of neutrophils by ET-1.

Phosphoramidon is a non-specific endothelin converting-enzyme inhibitor that inactivates ECE-1 as well as ECE-2. BQ-123 is a modified peptide, which must be administered parenterally. RNA knockdown requires hydrodynamic transfection.

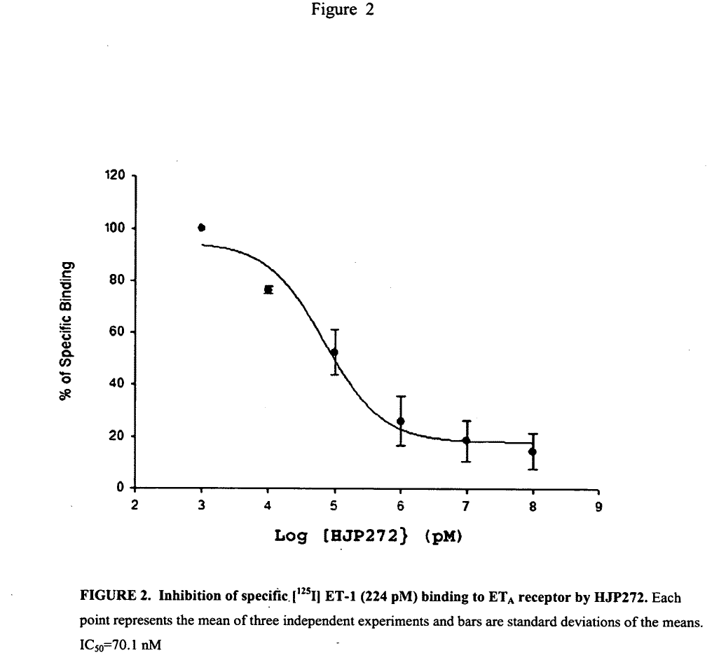

We have synthesized a series of compounds, the prototype of which is 3-(3-carboxybenzyl)-1-(6-ethylbenzo[d][1,3]dioxol-5-yl)methyl)-6-hydroxy-4-oxo-1,4-dihydroquinoline-2-carboxylic acid (HJP272) FIG. 2.

This novel, non-peptide, potentially orally active compound was found to bind the ET_{A} receptor selectively. Furthermore, the compound effectively controlled infection-associated preterm labor and delivery in our mouse model.

In addition, ET-1 plus cigarette smoke triggered selective recruitment of alveolar neutrophils was reversed by concomitant treatment with HJP-272, suggesting a possible role for this type of agent in reducing smoking related acute lung injury.

Due to the broad range of disorders with pathology now known to involve ET-1, the compounds may be useful in the treatment of many disorders, ranging from vascular disease to cancer.

### Methods

### A. Synthetic Methods

Synthesis of HJP272, the prototype compound with the formula of 1a (R, ═H, R′C₁H₅, R″═H)

According to the invention we provide the method of synthesis of the prototype compound 1-(5-1-(5-Hydroxy-2-nitrophenyl)ethanone

3-Hydroxyacetophenone, 80 g (0.588 mole) was dissolved in 320 ml of glacial acetic acid and placed in 3-neck flask, with one neck fitted with thermometer and other with condenser. With efficient stirring, at 70° C., 47.8 ml (0.676 mole) of nitric acid (d 1.4) is added drop wise carefully assuring that temperature is not deviating from 70° C. by more than ±2° C. After addition, reaction was stirred for another 2 hrs at 65-70° C. and then poured into around 300 ml of ice-water and kept overnight in the refrigerator. Next day precipitates were filtered and allowed to dry. Precipitates were then taken in to 500 ml round bottom flask and were boiled in 200 ml of benzene for 1 hour. Benzene was decanted while hot leaving sticky black residue in the round bottom flask and then allowed to cool to room temperature resulting in 38.4 g of mixture of nitration products. Column chromatography (CHCl₃: EtOAc; 85:15) resulted in 14.3 g (17.9%) of the desired 1-(5-hydroxy-2-nitrophenyl)ethanone regioisomer, 8.4 g (10.4%) of 1-(5-hydroxy-6-nitrophenyl)ethanone and 1.5 g (1.9%) of 1-(5-hydroxy-4-nitrophenyl)ethanone.

mp. 146-148° C. (Klinke et. al. 1961, 148-149° C.), TLC (80% CHCl₃: 20% EtOAc) R_{f}=0.5100.

MS: m/z 181 (M⁺); 166 [(M-CH₃)⁺, C₇H₄NO₄]; 139 [(M-CH₂═C═O)⁺, C₆H₅NO₃]; 122 (139-OH.)⁺; 109 [(139-NO)⁺, C₆H₅O₂]; 92 (139-HNO₂⁺, C₆H₄O); 80 C₅H₄O; C₆H₅⁺; 63 C₅H₃⁺, 53 C₄H₅⁺, 52 C₄H₄⁺; 43 CH₃-+C═O (base peak).

IR: 1711 cm⁻ (ketone C═O), 1523 and 1341 cm⁻¹ (aromatic nitro).

¹H-NMR (DMSO-d₆, 8 ppm): a: 2.49, s, 3H; b: 6.85, s, 1H; c: 6.99, d, 1H, J_{cd}=7.8 Hz; d: 8.09, d, 1H, J=7.8 Hz; e: 11.39, s, 1H.

¹³C-NMR (DMSO-d₆, δ ppm): C₁: 30.24, C₂: 113.37, C₃: 116.55, C₄: 127.48, C₅: 136.32, C₆: 141.10, C₇: 163.55, C₈: 200.22.

### (E)-3-(3-(5-Hydroxy-2-nitrophenyl)-3-oxoprop-1-enyl)benzoic acid

1-(5-Hydroxy-2-nitrophenyl)ethanone, 13 g (0.0718 mole) was dissolved in 150 ml of methanol and placed in 500 ml round-bottomed flask. To this 23 ml (3 equiv.) of 10 N NaOH was added and allowed to stir at room temperature for 15 mins, followed by addition of 10.77 g (0.0718 mole) of 3-carboxybenzaldehyde. Reaction mixture was refluxed for 12 hrs and monitored on TLC. The reaction mixture was allowed to cool and neutralized with 23 ml of 10 N HCl, resulting in the formation of a precipitate. The precipitate was filtered, washed thoroughly with water and allowed to dry, to give 21.2 g of crude product. Recrystallization from methanol-water (5%) resulted in 19.4 g (86.3%) of pure product,

m.p. 253-254° C., TLC (90% CH₂Cl₂: 10% EtOAc) R_{f}=0.306.

MS: m/z 313 (M⁺), 296, 284, 268, 250, 163 (base peak), 150, 135, 131, 120, 107, 92, 91, 77, 65, 51.

IR: 3408 and 3215 cm⁻¹ (—OH), 1702 cm⁻¹ (α, β unsaturated ketone C═O), 1627 cm⁻¹ (aromatic carboxylic acid C═O), 1575 and 1332 cm⁻¹ (aromatic nitro).

¹H-NMR (DMSO-d₆, δ ppm): a: 6.84, d, 1H, J=2.2 Hz; b: 7.07, dd, 1H, J_{bh}=8.8; c: 7.26, d, 1H, J_{cd}=16.4 Hz; d: 7.46, d, 1H, J_{dc}=16.3 Hz; e: 7.566, t, 1H, J=7.7 Hz; f: 7.99, d, 1H, J_{fb}=8 Hz; g: 8.04, d, 1H, J_{ge}=7.6 Hz; h: 8.19, d, 1H, Jₕₑ=9.2; i: 8.23, s, 1H; j: 12.32 (exchangeable with D₂O), s, 2H.

¹³C-NMR (DMSO-d₆, δ ppm): C₁: 114.73, C₂: 116.82, C₃: 127.27, C₄: 127.72, C₅: 129.30, C₆: 129.90, C₇: 131.35, C₈: 131.64, C₉: 132.30, C₁₀: 134.51, C₁₁: 137.38, C₁₂: 139.06, C₁₃: 144.28, C₁₄: 163.45, C₁₅: 166.84, C₁₆: 192.77.

Anal.: Calc. for C₁₆H₁₁NO₆: C, 61.35; H, 3.54; N, 4.47. Found: C, 61.06; H, 3.54; N, 4.41.

### (E)-Ethyl 3-(3-(5-Hydroxy-2-nitrophenyl)-3-oxoprop-1-enyl)benzoate

(E)-3-(3-(5-Hydroxy-2-nitrophenyl)-3-oxoprop-1-enyl)benzoic acid, 25 g (0.0799 mole) was dissolved in 450 ml of anhydrous ethanol (dried over molecular sieves overnight). Anhydrous hydrochloric acid gas was bubbled in the reaction mixture at 0° C. and allowed to stir overnight at room temperature. At the end of reaction (disappearance of starting material on TLC) ethanol and excess HCl gas were evaporated on rotary evaporation to get crude product. Recrystallization from Hexane-EtOAc resulted in 26.0 g (95%) of white crystalline product, m.p. 157-159° C., TLC (90% CHCl₃: 10% EtOAc) R_{f}=0.414.

MS: m/z 341 (M⁺), 312 (M-C₂H₅)⁺, 296 (M-O C₂H₅)⁺, 268 (M-COO C₂H₅)⁺, 250 (296-NO₂)⁺, 196 C₉H₆NO₄, 179 C₈H₅NO₄, 163 (base peak, C₁₀H₁₁O₂), 150 C₇H₄NO₃, 135 [(163-C₂H₄)⁺, C₈H₇O₂], 120 (150-NO)⁺, C₇H₄O₂, 107 C₇H₇₀, 92 C₆H₄O, 91 C₇H₇⁺, 77 C₆H₅⁺, 65 C₅H₅⁺, 51 C₄H₃⁺.

IR: 3376 cm⁻¹ (—OH), 1714 cm⁻¹ (aromatic ester, C═O), 1672 cm⁻¹ (α, β unsaturated ketone C═O), 1590 and 1340 cm⁻¹ (aromatic nitro).

¹H-NMR (DMSO-d₆, δ ppm): a: 1.33, t, 3H, J_{ab}=7.1 Hz; b: 4.33, q, 2H, J_{ba}=7 Hz; c: 6.85, d, 1H, J=2.2 Hz; d: 7.08, dd, 1H, J_{dj}=9.0; e: 7.27, d, 1H, J_{cf}=16.4 Hz; f: 7.46, d, 1H, J_{dc}=16.3 Hz; g: 7.58, t, 1H, J=7.7 Hz; h: 8.00, d, 1H, J_{hg}=7.6 Hz; i: 8.08, d, 1H, J_{ig}=7.5 Hz; j: 8.19, d, 1H, J_{jd}=9.1; k: 8.22, s, 1H; l: 11.42 (exchangeable with D₂O), s, 1H.

¹³C-NMR (DMSO-d₆, 8 ppm): C₁: 14.12, C₂: 60.95, C₃: 114.71, C₄: 116.79, C₅: 127.48, C₆: 127.70, C₇: 129.39, C₈: 129.68, C₉: 130.70, C₁₀: 131.09, C₁₁: 132.49, C₁₂: 134.65, C₁₃: 137.39, C₁₄: 138.97, C₁₅: 144.12, C₁₆: 163.38, C₁₇: 165.22, C₁₈: 192.76.

Anal.: Calc. for C₁₈H₁₅NO₆: C, 63.28; H, 4.43; N, 4.10. Found: C, 63.28; H, 4.46; N, 4.14.

### (E)-Ethyl 3-(3-(5-(methoxymethoxy)-2-nitrophenyl)-3-oxoprop-1-enyl)benzoate

Sodium hydride dispersion (60% in mineral oil), 3.03 g equivalent to 1.81 g of NaH (0.0756 mol, 1.2 equiv.) was weighed and placed in the three-neck flask under nitrogen. Mineral oil was washed out with 10 ml of hexane for three times followed by addition of 15 ml of dry dimethyl formamide (dried over molecular sieves overnight). Three-neck flask was then kept in the ice-water bath. When the temperature of the reaction has reached 0° C., 21.5 g (0.0630 mole) of (E)-ethyl 3-(3-(5-hydroxy-2-nitrophenyl)-3-oxoprop-1-enyl)benzoate dissolved in 50 ml of dry DMF was added drop wise with stirring. After addition the ice-bath was removed and the reaction was allowed to stir at room temperature for 20 mins. Methoxymethyl chloride, 6.05 g (0.0756 mol, 1.2 equiv.) in 15 ml of dry DMF was added drop wise at 0° C. followed by addition of 20 g of potassium carbonate to make pH 8. Reaction was allowed to stir at room temperature for 4 hrs. and then poured into 400 ml of ice water. Mixture was then transferred to the 1000 ml separatory funnel and extracted with 100 ml of ethyl ether for three times. Combined ether solution was then dried over magnesium sulfate, filtered and evaporated to get 23.1 g of crude product. Recrystallization from ethyl acetate-hexane (2%) resulted in 21.6 g (89%) of pure product,

m.p. 88-91° C. TLC (60% n-hexane: 40% EtOAc) R_{f}=0.357.

MS: m/z 385 (M⁻⁺), 356 (M-C₂H₅⁻)⁺, 340 [(M-45), (M-CH₂—OCH₃)⁺ or (M-CH₃CH₂O⁻)⁺], 312 (M-73) (M-COOC₂H₅)⁺, 294 [(M-91) (340-NO₂)⁺], 236 (M-149) C₁₁H₁₀NO₅, 207 (M-178), 206 (236-NO)⁺, 190 (236-NO₂)⁺, 176 C₁₁H₁₂O₂ CH₂═CH—C₆H₄—COOC₂H₅, 163 (base peak, +CH₂—C₆H₄—COOC₂H₅, C₁₀H₁₁O₂), 149 C₉H₉O₂, 133 [(163-CH₂O)⁺, 129 (157-CO), 102 (176-HCOOC₂H₅)⁺ and/or (163-OCH₂OCH₃)⁺, 91 C₇H₇⁺, 77 C₆H₅⁺, 65 C₅H₅⁺, 59 CH₃OCH₂—O⁺, 45 CH₃—⁺O═CH₂.

IR: 1719 cm⁻¹ (aromatic ester, C═O), 1648 cm⁻¹ (α,β unsaturated ketone C═O), 1518 and 1340 cm⁻¹ (aromatic nitro).

¹H-NMR (CDCl₃, δ ppm): a: 1.40, t, 3H, J_{ac}=7.2 Hz; b: 3.51, s, 3H; c: 4.33, q, 2H, J_{ca}=7.1 Hz; d: 5.29, s, 2H; e: 7.03, d, 1H, Jₑₕ=16.3 Hz; f: 7.05, d, 1H, J=2.6 Hz; g: 7.27, dd, 1H, J_{mg}=9.3 Hz; h: 7.27, d, 1H, Jₕₑ=16.4 Hz; i: 7.47, t, 1H, J=7.8 Hz; j: 7.70, d, 1H, Jⱼᵢ=7.8 Hz; k: 8.06, d, 1H, Jₖᵢ=7.7 Hz; l: 8.14, s, 1H; m: 8.22, d, 1H, J_{mg}=9.1 Hz.

¹³C-NMR (CDCl₃, δ ppm): C₁: 14.50, C₂: 56.89, C₃: 61.54, C₄: 94.76, C₅: 115.86, C₆: 117.20, C₇: 127.26, C₈: 127.54, C₉: 129.29, C₁₀: 129.81, C₁₁: 131.55, C₁₂: 131.86, C₁₃: 132.43, C₁₄: 134.53, C₁₅: 138.97, C₁₆: 140.16, C₁₇: 144.65, C₁₈: 162.05, C₁₉: 166.01, C₂₀: 192.71.

Anal.: Calc. for C₂₀H₁₉NO₇: C, 62.33; H, 4.97; N, 3.63. Found: C, 62.11; H, 4.94; N, 3.64.

### Ethyl 3-(3-(2-amino-5-(methoxymethoxy)phenyl)-3-oxopropyl)benzoate

(E)-Ethyl 3-(3-(5-(methoxymethoxy)-2-nitrophenyl)-3-oxoprop-1-enyl)benzoate, 21.0 g (0.0545 mole) was dissolved in 80 ml of dry ethanol and placed in the hydrogenation bomb. To the reaction solution, 100 mg of platinum oxide was added and the catalyst was activated after two cycles of vacuum-nitrogen. Hydrogenation was done at 45 psi of hydrogen for 30 mins with stirring at room temperature. Five drops of acetic acid were added followed by hydrogenation at 45 psi hydrogen for another 15 mins. At the end of reaction catalyst was carefully filtered over celite under nitrogen followed by evaporation of solvent on the rotary evaporation to get

19.0 g of thick oil. Column chromatography (60% Hexane: 40% EtOAc) resulted in 16.1 g (82.7%) of pure product. TLC (60% n-hexane: 40% EtOAc) R_{f}=0.643.

MS: m/z 357 (M⁺), 327 (M-30) (M-CH₂O)⁺, 325 (M-32) (M-CH₃OH)⁺, 312 [(M-45), (M-OC₂H₅)+ or (M-CH₂OCH₃)⁺], 266 (M-91), 176 [(M-181), C₁₁H₁₂O₂, CH₂═CHC₆H₄COOC₂H₅), 162 C₁₀H₁₀O₂, 150 (C₉H₁₀O₂, C₆H₅COOC₂H₅), 136 (C₈H₈O₂, CH₃OCH₂OC₆H₃), 119 C₇H₅NO, 108 C₆H₆NO, 92 C₆H₄O, 91 C₇H₇⁺, 79 C₆H₇⁺, 77 C₆H₅⁺, 65 C₅H₅⁺, 52 C₄H₄, 45 (base peak, CH₃—⁺O═CH₂).

IR: 3466 and 3350 cm⁻ (primary amine N—H), 1716 cm⁻¹ (aromatic ester, C═O), 1650 cm⁻¹ (ketone C═O).

¹H-NMR (CDCl₃, δ ppm): a: 1.39, t, 3H, Jₐₑ=7.1 Hz; b: 3.08, t, 2H, J_{bc}=7.9 Hz; c: 3.08, t, 2H, J_{bc b}=7.2 Hz; d: 3.48, s, 3H; e: 4.38, q, 2H, Jₑₐ=7.1 Hz; f: 5.06, s, 2H; g: 6.04 (exchangeable with D₂O), s, 2H; h: 6.61, d, 1H, Jₕᵢ=8.9 Hz; i: 7.06, dd, 1H, Jⱼₕ=8.9 Hz; j: 7.36, t, J=7.6 Hz; k: 7.4, s, 1H; l: 7.45, d, 1H, Jₗⱼ=7.6 Hz; m: 7.89, d, 1H, Jₘⱼ=7.7 Hz; n: 7.94, s, 1H.

¹³C-NMR (CDCl₃, δ ppm): C₁: 14.53, C₂: 30.50, C₃: 40.98, C₄: 56.08, C₅: 61.13, C₆: 96.04, C₇: 117.85, C₈: 118.04, C₉: 118.64, C₁₀: 125.46, C₁₁: 127.56, C₁₂: 128.68, C₁₃: 129.68, C₁₄: 130.92, C₁₅: 133.27, C₁₆: 141.93, C₁₇: 146.25, C₁₈: 147.65, C₁₉: 166.87, C₂₀: 200.78.

### 5-Ethylbenzo[d][1,3]dioxole

Hydrazine hydrate (55% hydrazine), 40 ml (0.683 mole; 2.8 equiv.) was placed in 500 ml of round bottom flask containing 150 ml of ethanol. To this 40 g (0.244 mole) of 1-(benzo[d][1,3]dioxol-5-yl)ethanone was added and the reaction mixture refluxed for 6 hours. GC-MS showed a single peak of M⁺178 m/z for hydrazone (m.p. 89-93° C.). Ethanol was evaporated by rotary evaporation and 50 ml of ethylene glycol was added to the flask containing crude hydrazone. The solution was then transferred to 3-neck flask with one neck fitted with condenser and other with nitrogen inlet. To this add 50 g of potassium hydroxide was added and the mixture was heated at 160° C. under nitrogen for 8 hours. After the completion of reaction it was allowed to cool and water was added to it. The resultant mixture was extracted with ether (3×150 ml). The organic layer was then washed with 1N HCl (2×150 ml) followed by water (2×50 ml), dried over MgSO₄ and evaporated to get 28.6 g of crude product. Vacuum distillation of crude product

130° C. and 27 mmHg resulted in 23.2 g (63.4%) of pure product (Kumar BR 2006).

MS: m/z 150 (M⁺), 135 (base peak, M⁺-CH₃), 121 (M⁺-C₂H₅), 105 (135-CH₂═O)⁺, 91 C₇H₇⁺, 79 C₅H₃O⁺, 77 C₆H₅⁺, 65 C₅H₅⁺, 63 C₅H₃⁺, 51 C₄H₃⁺.

¹H-NMR (CDCl₃, δ ppm): a: 1.19, t, 3H, J_{ab}=7.6 Hz; b: 2.56, q, 2H, J_{ba}=7.2 Hz; c: 5.89, s, 2H; d: 6.63, d, 1H, J_{df}=7.5 Hz; e: 6.69, s, 1H; f: 6.72, d, 1H, J_{fd}=7.8 Hz.

¹³C-NMR (CDCl₃, δ ppm): C₁: 16.15, C₂: 28.24, C₃: 100.87, C₄: 108.27, C₅: 108.60, C₆: 120.58, C₇: 138.39, C₈: 145.59, C₉: 147.70.

### 6-Ethylbenzo[d][1,3]dioxole-5-carbaldehyde

5-Ethylbenzo[d][1,3]dioxole, 22 g (0.146 mole) was stirred with 50 ml of dichloromethane in a 500 ml round bottom flask. To this 33.73 g (0.292 mole; 2 equiv.) of α,α-dichloromethyl methyl ether in 50 ml of dichloromethane was added dropwise at 0° C. with stirring. After 15 minutes 33.38 g (0.176 mole; 1.2 equiv.) of titanium tetrachloride in 50 ml of dichloromethane was added dropwise at 0° C. After complete addition remove the ice bath and the reaction was stirred at room temperature for 1 hour. At the end reaction mixture was poured into 250 ml of ice-water and extracted with diethyl ether (3×75 ml) and ethyl acetate (3×75 ml). The combined organic extracts were washed with 100 ml of brine followed by aqueous sodium bicarbonate (3×100 ml). After drying over MgSO₄, the organic extract was concentrated over rotary evaporation to get 25.9 g of crude product. Column chromatography of the crude product (90% Hexane: 10% EtOAc) resulted in 22.6 g (86.6%) of pure product.

TLC (90% Hexane: 10% EtOAc) R_{f}=0.397.

MS: m/z 178 (base peak, M⁺), 177 (M-H), 163 (M⁺-CH₃), 161 (M-CH₃—H₂)⁺, 149 (M-C₂H₅)⁺ or (M-CHO)⁺, 135 (M-CH₃—CO)⁺, 131 (161-CH₂O)⁺, 119 (149-CH₂O)⁺, 105 C₆H₅CO⁺, 91 C₇H₇⁺, 79 C₅H₃O⁺, 77 C₆H₅⁺, 65 C₅H₅⁺, 51 C₄H₃⁺.

IR: 1699 cm⁻¹ (aromatic aldehyde, C═O)

¹H-NMR (CDCl₃, δ ppm): a: 1.26, t, 3H, J_{ab}=7.6 Hz; b: 2.98, q, 2H, J_{ba}=7.6 Hz; c: 6.02, s, 2H; d: 6.72, s, 1H; e: 7.30, s, 1H; f: 10.17, s, 1H.

¹³C-NMR (CDCl₃, δ ppm): C₁: 17.21, C₂: 25.38, C₃: 101.37, C₄: 108.5, C₅: 109.87, C₆: 128.0, C₇: 145.08, C₈: 145.74, C₉: 152.69, C₁₀: 189.52.

### Ethyl 3-(3-(2-((6-ethylbenzo[d][1,3]dioxol-5-yl)methylamino)-5-(methoxymethoxy)phenyl)-3-oxopropyl)benzoate

In a 500 ml round bottom flask 16 g (0.0448 mole) of aminoketone, ethyl 3-(3-(2-amino-5-(methoxymethoxy)phenyl)-3-oxopropyl)benzoate was dissolved in 150 ml of dichloroethane. To this 12 g (0.0672 mole; 1.5 equiv.) of 6-ethylbenzo[d][1,3]dioxole-5-carbaldehyde in 25 ml of dichloromethane was added. After stirring for 15 minutes 19 g of sodium triacetoxyborohydride (0.0896 mole; 2 equiv.) was added and the reaction was stirred at room temperature for 5 hours. At the end reaction mixture was poured into 250 ml of ice-water and extracted with diethyl ether (3×100 ml). After drying over MgSO₄, the organic extract was concentrated over rotary evaporation to get 19.3 g of oil. After triturating the oil with 4 ml of isopropyl alcohol it resulted in 17.5 g of solid crude product, m.p. 81-87° C. Column chromatography of the crude product (70% Hexane: 30% EtOAc) resulted in 16.2 g (70.6%) of pure product.

m.p. 93-94° C., TLC (80% Hexane: 20% EtOAc) R_{f}=0.58.

MS: m/z 519 (M⁺), 490 (M-C₂H₅)⁺, 488 (M-OCH₃)⁺, 474 (M-OC₂H₅)⁺ or (M-CH₃OCH₂)⁺, 357 (M-162)⁺, 356 (M-163 or M-C₁₀H₁₁O₂)⁺, 327 (357-CHO)⁺, 312 (357-CH₃OCH₂)⁺, 163 (base peak, C₁₀H₁₀O₂), 133 [(163-CH₂O)⁺, C₉H₉O], 105 [(133-C₂H₄)⁺, C₇H₅O], 91 C₇H₇⁺, 79 C₆H₇⁺, 77 C₆H₅⁺, 45 CH₃—⁺O═CH₂.

IR: 3329 cm⁻¹ (secondary amine N—H), 1716 cm⁻¹ (aromatic ester, C═O), 1644 cm⁻¹ (ketone C═O).

¹H-NMR (CDCl₃, δ ppm): a: 1.21, t, 3H, J_{ac}=7.5 Hz; b: 1.39, t, 3H, J_{bh}=7.1 Hz; c: 2.61, q, 2H, J_{ca}=7.6 Hz; d: 3.07, t, 2H, J_{de}=7.4 Hz; e: 3.28, t, 2H, J_{ed}=8.0 Hz; f: 3.48, s, 3H; g: 4.39, d, 2H, J=5.2 Hz; h: 4.38, q, 2H, J_{hb}=7.1 Hz; i: 5.06, s, 2H; j: 5.90, s, 2H; k: 6.59, d, 2H, Jₖₙ=9.2 Hz; l: 6.72, s, 1H; m: 6.78, s, 1H; n: 7.13, dd, 1H, Jₖₙ=9.1 Hz; o: 7.36, t, 1H, J=7.5 Hz; p: 7.44, d, 1H, Jₚₒ=7.4 Hz; q: 7.47, s, 1H; r: 7.89, d, 1H, Jᵣₒ=7.6 Hz; s: 7.94, s, 1H; t: 8.8 (exchangeable with D₂O), s, 1H.

¹³C-NMR (CDCl₃, δ ppm): C₁: 14.56, C₂: 15.53, C₃: 25.53, C₄: 30.67, C₅: 41.01, C₆: 44.82, C₇: 56.10, C₈: 61.153, C₉: 96.17, C₁₀: 101.01, C₁₁: 108.67, C₁₂: 109.17, C₁₃: 113.35, C₁₄: 117.29, C₁₅: 119.22, C₁₆: 126.01, C₁₇: 127.60, C₁₈: 128.71, C₁₉: 128.99, C₂₀: 129.69, C₂₁: 130.95, C₂₂: 133.27, C₂₃: 135.74, C₂₄: 141.95, C₂₅: 146.01, C₂₆: 146.55, C₂₇: 147.08, C₂₈: 147.19, C₂₉: 166.89, C₃₀: 200.89.

Anal.: Calc. for C₃₀H₃₃NO₇: C, 69.35; H, 6.40; N, 2.70. Found: C, 69.07; H, 6.46; N, 2.71.

### Ethyl 3-(3-(2-(2-ethoxy-N-((6-ethylbenzo[d][1,3]dioxol-5-yl)methyl)-2-oxoacetamido)-5-(methoxymethoxy)phenyl)-3-oxopropyl)benzoate

In a solution of 16 g (0.031 mole) of secondary amine in 60 ml of N,N-dimethyl formamide (dried over molecular sieves overnight), 3.1 g (0.04 mole) of triethylamine was added dropwise at 0° C. The mixture was allowed to stir at room temperature for 15 minutes followed by addition of 5.44 g (0.04 moles) of ethyl oxalyl chloride in 15 ml of DMF. The reaction was stirred for 4 hours and then poured into 200 ml of ice-water followed by extraction with 100 ml of ethyl acetate for three times. Organic extract was dried over MgSO₄ and evaporated to get 18.4 g of crude oil. Column chromatography of the oil (60% Hexane: 40% EtOAc) resulted in 18.0 g (94.3%) of pure product as colorless oil.

TLC (60% Hexane: 40% EtOAc) R_{f}=0.373.

MS: m/z 619 (M⁺), 602 (M-17, M-OH)⁺, 601 (M-H₂O)⁺, 556 (M-H₂O—C₂H₅)⁺, 546 (M-73, M-COOC₂H₅)⁺, 545 (M-74, M-HCOOC₂H₅)⁺, 518 (M-101, M-C(O)—COOC₂H₅)⁺, 438 (M-181, M-H₂O—CH₂C₆H₄COOC₂H₅), 364 (438-COOC₂H₅)⁺, 163 (base peak, +CH₂C₆H₄COOC₂H₅, C₁₀H₁₁O₂), 133 [(163-CH₂O)⁺, C₉H₉O], 105 [(133-C₂H₄)⁺, C₇H₅O], 91 C₇H₇⁺, 79 C₆H₇⁺, 77 C₆H₅⁺, 45 CH₃—⁺O═CH₂.

IR: 2965 and 2934 cm⁻¹ (alkyl), 1739 cm⁻¹ (oxalyl ester, C═O) 1721 cm⁻¹ (aromatic ester, C═O), 1702 cm⁻¹ (amide, C═O), 1676 cm⁻¹ (ketone C═O).

¹H-NMR (CDCl₃, δ ppm): a: 0.97, t, 3H, J_{ad}=7.5 Hz; b: 1.02, t, 3H, J_{bh}=7.2 Hz; c: 1.40, t, 3H, J_{ci}=7.1 Hz; d: 2.28, q, 2H, J_{da}=7.7 Hz; e: 3.05-3.15, m, 4H; f: 3.44, s, 3H; g: 4.02, q, 2H, J_{gb}=7.0 Hz; h: 4.37, q, 2H, J_{hc}=7.1 Hz; i: 4.47, d, 1H, J=14.5 Hz, j: 5.14, s, 2H; k: 5.29, d, 1H, J=14.5 Hz; l: 5.87, d, 2H; m: 6.59, s, 1H; n: 6.65, s, 1H; o: 6.71, d, 1H, Jₒₚ=8.7; p: 6.95, dd, 1H, Jₚₒ=8.6 Hz; q: 7.18, d, 1H, J=2.6 Hz; r: 7.37, t, 1H, J=7.6 Hz; s: 7.45, d, 1H, Jₛᵣ=7.5 Hz; t: 7.89, d, 1H; J=7.6 Hz; u: 7.94, s, 1H.

¹³C-NMR (CDCl₃, δ ppm): C₁: 13.91, C₂: 14.53, C₃: 15.68, C₄: 25.35, C₅: 29.68, C₆: 42.79, C₇: 49.14, C₈: 56.42, C₉: 61.14, C₁₀: 61.78, C₁₁: 94.64, C₁₂: 101.09, C₁₃: 108.71, C₁₄: 110.97, C₁₅: 116.61, C₁₆: 118.77, C₁₇: 126.20, C₁₈: 127.65, C₁₉: 128.75, C₂₀: 129.60, C₂₁: 130.21, C₂₂: 130.96, C₂₃: 133.07, C₂₄: 133.33, C₂₅: 137.53, C₂₆: 139.30, C₂₇: 141.50, C₂₈: 145.83, C₂₉: 147.60, C₃₀: 157.34, C₃₁: 161.18, C₃₂: 162.32, C₃₃: 166.84, C₃₄: 200.24.

### Ethyl 3-(3-(ethoxycarbonyl)benzyl)-1-((6-ethylbenzo[d][1,3]dioxol-5-yl)methyl)-6-(methoxymethoxy)-4-oxo-1,4-dihydroquinoline-2-carboxylate

Dissolve 18.2 g (0.0294 mole) of Ethyl 3-(3-(2-(2-ethoxy-N-((6-ethylbenzo[d][1,3]dioxol-5-yl)methyl)-2-oxoacetamido)-5-(methoxymethoxy)phenyl)-3-oxopropyl)benzoate in 100 ml of ethanol in a 500 ml round bottom flask. To this 5.37 g (0.0353 mole, 1.2 equiv.) of 1,8-diazabicyclo[5.4.0]undec-7-ene DBU) was added dropwise and stirred at room temperature for 2 hours. After removing the solvent on rotary evaporation, the residue was dissolved in 150 ml of ethyl acetate and washed with 100 ml of water for three times. Organic extract was dried over MgSO₄ and evaporated to get 17.2 g of crude product, m.p. 89-96° C. Recrystallization from EtOAc-hexane (95:5) resulted in 16.3 g (92%) of pure product.

m.p.=99-101° C., TLC (50% Hexane: 50% EtOAc) R_{f}=0.776.

MS: m/z 601 (M⁺), 556 [(M-CH₂—OCH₃)+ or (M-CH₃CH₂O)⁺], 528 [(M-73)⁺, M-COOC₂H₅)⁺], 438 (M-163, M-C₁₀H₁₁O₂)⁺, 410 [(438-CO)⁺ or (438-C₂H₄)⁺], 364 (438-HCOOC₂H₅)⁺, 163 (base peak, +CH₂C₆H₄COOC₂H₅, C₁₀H₁₁O₂), 133 [(163-CH₂O)⁺, C₉H₉O], 105 [(133-CO) or 133-C₂H₄)⁺, C₇H₅O], 91 C₇H₇⁺, 79 C₆H₇⁺, 77 C₆H₅⁺, 45 CH₃—⁺O═CH₂.

IR: 2964 and 2933 cm⁻¹ (alkyl), 1730 cm⁻¹ (α β unsaturated ester, C═O), 1714 cm⁻¹ (aromatic ester, C═O), 1597 cm⁻¹ (quinol-4-one, C═O).

¹H-NMR (CDCl₃, δ ppm): a: 1.05, t, 3H, J_{ad}=7.1 Hz; b: 1.27, t, 3H, J_{bg}=7.5 Hz; c: 1.36, t, 3H, J_{ch}=7.1 Hz; d: 2.64, q, 2H, J_{da}=7.5 Hz; e: 3.46, s, 3H; f: 3.98, s, 2H; g: 4.24, q, 2H, J_{gb}=7.1 Hz; h: 4.34, q, 2H, J_{hc}=7.1 Hz; i: 5.19, s, 2H; j: 5.23, s, 2H; k: 5.86, s, 2H; l: 6.23, d, 1H; m: 6.74, s, 1H; n: 7.10, d, 1H, Jₙₒ=9.3 Hz; o: 7.24, dd, 1H, Jₒₙ=9.3 Hz; p: 7.31, t, 1H, J=7.7 Hz; q: 7.59, d, 1H, J=7.5 Hz; r: 7.84, d, 1H, J=7.7 Hz; s: 7.99, s, 1H; t: 8.03, d, 1H; J=2.7 Hz.

¹³C-NMR (CDCl₃, δ ppm): C₁: 13.68, C₂: 14.53, C₃: 14.77, C₄: 25.61, C₅: 32.46, C₆: 51.35, C₇: 56.42, C₈: 60.93, C₉: 62.94, C₁₀: 94.84, C₁₁: 101.25, C₁₂: 106.50, C₁₃: 109.07, C₁₄: 110.80, C₁₅: 117.94, C₁₆: 118.23, C₁₇: 124.01, C₁₈: 125.54, C₁₉: 127.44, C₂₀: 127.51; C₂₁: 128.44, C₂₂: 129.86, C₂₃: 130.46, C₂₄: 133.59, C₂₅: 133.62, C₂₆: 135.46, C₂₇: 140.54, C₂₈: 144.41, C₂₉: 146.56, C₃₀: 147.48, C₃₁: 153.97, C₃₂: 163.93, C₃₃: 166.99, C₃₄: 176.73.

Anal.: Calc. for C₃₄H₃₅NO₉: C, 67.87; H, 5.86; N, 2.33. Found: C, 67.49; H, 5.98; N, 2.34.

### Ethyl 3-(3-(ethoxycarbonyl)benzyl)-1-((6-ethylbenzo[d][1,3]dioxol-5-yl)methyl)-6-hydroxy-4-oxo-1,4-dihydroquinoline-2-carboxylate 1a, (R, ═H, R′, R″═C₂H₅)

Ethyl 3-(3-(ethoxycarbonyl)benzyl)-1-((6-ethylbenzo[d][1,3]dioxol-5-yl)methyl)-6-(methoxymethoxy)-4-oxo-1,4-dihydroquinoline-2-carboxylate was dissolved in 350 ml of anhydrous ethanol (dried over molecular sieves overnight). Anhydrous hydrochloric acid gas was bubbled in the reaction mixture at 0° C. and allowed to stir overnight at room temperature. At the end of reaction (disappearance of starting material on TLC) ethanol and excess HCl gas were evaporated on rotary evaporation to get crude product. Recrystallization from ethanol-water (98:2) resulted in 12.45 g (82.5%) of pure product.

m.p.=232-234° C., TLC (50% Hexane: 50% EtOAc) R_{f}=0.52.

MS: m/z 557 (M⁺), 528 (M-C₂H₅)⁺, 527 (M-CH₂O⁺), 512 (M-C₂H₅O)⁺, 484 (M-COOC₂H₅)⁺, 408 (C₂₃H₂₂NO₆), 394 [(M-163)⁺, (M-C₁₀H₁₁O₂)⁺], 366 (394-CO)⁺, 320 [(394-HCOOC₂H₅)⁺, C₁₉H₁₄NO₄], 248 (C₁₆H₁₀NO₂), 220 [(248-CO)⁺, C₁₅H₁₀NO], 163 (base peak, +CH₂C₆H₄COOC₂H₅, C₁₀H₁₁O₂), 133 [(163-CH₂O)⁺, C₉H₉O], 105 [(133-CO) or 133-C₂H₄)⁺, C₇H₅O], 91 C₇H₇⁺, 79 C₆H₇⁺, 77 C₆H₅⁺, 55 C₄H₇⁺.

IR: 3258 cm⁻¹ (phenolic —OH), 1727 cm⁻¹ (α β unsaturated ester, C═O), 1714 cm⁻¹ (aromatic ester, C═O), 1599 cm⁻¹ (quinol-4-one, C═O).

¹H-NMR (DMSO-d₆, δ ppm): a: 0.99, t, 3H, J_{ad}=6.6 Hz; b: 1.19, t, 3H, J_{bf}=6.9 Hz; c: 1.29, t, 3H, J_{cf}=6.8 Hz; d: 2.67, q, 2H, J_{da}=7.1 Hz; e: 3.84, s, 2H; f: 4.27, q, 4H, J=6.7 Hz; g: 5.30, s, 2H; h: 5.90, s, 2H; i: 5.93, s, 1H; j: 6.88, s, 1H; k: 7.18, d, 1H, Jₖₗ=7.1 Hz; l: 7.26, d, 1H, Jₗₖ=9.0 Hz; m: 7.39, t, 1H, J=7.4 Hz; n: 7.53, d, 1H, Jₙₘ=8.8 Hz; o: 7.55, s, 1H; p: 7.76, d, 1H, Jₚₘ=6.8 Hz; q: 7.86, s, 1H; r: 9.97 (exchangeable with D₂O), s, 1H.

¹³C-NMR (DMSO-d₆, δ ppm): C₁: 13.17, C₂: 14.13, C₃: 14.46, C₄: 24.41, C₅: 31.58, C₆: 50.50, C₇: 60.61, C₈: 62.75, C₉: 100.89, C₁₀: 105.15, C₁₁: 108.27, C₁₂: 108.79, C₁₃: 115.48, C₁₄: 119.24, C₁₅: 122.85, C₁₆: 126.08, C₁₇: 126.69, C₁₈: 126.75, C₁₉: 128.34, C₂₀: 128.86, C₂₁: 129.66, C₂₂: 132.94, C₂₃: 133.19, C₂₄: 133.99, C₂₅: 140.82, C₂₆: 143.90, C₂₇: 145.47, C₂₈: 146.52, C₂₉: 154.31, C₃₀: 163.14, C₃₁: 165.77, C₃₂: 174.99.

Anal.: Calc. for C₃₂H₃₁NO₈: C, 68.93; H, 5.60; N, 2.51. Found: C, 68.73; H, 5.68; N, 2.64.

### General Procedure for the Synthesis of 1,3,6-Trisubstituted-2-Ethylcarboxylate-Quinol-4-ones

Sodium hydride dispersion (60% in mineral oil), 50-60 mg equivalent to 30-36 mg of NaH (1.2-1.6 equiv.) was weighed and placed in the three-neck flask under nitrogen. Mineral oil was washed out with 5 ml of hexane for three times followed by addition of 5 ml of dry dimethyl formamide (dried over molecular sieves overnight). Three-neck flask was then kept in the ice-water bath. When the temperature of the reaction had reached 0° C., 0.4-0.45 g (0.7181 mmol) of ethyl 3-(3-(ethoxycarbonyl)benzyl)-1-((6-ethylbenzo[d][1,3]dioxol-5-yl)methyl)-6-hydroxy-4-oxo-1,4-dihydroquinoline-2-carboxylate dissolved in 10 ml of dry DMF was added dropwise with stirring. After addition the ice-bath was removed and the reaction was allowed to stir at room temperature for 20 mins. Appropriate alkyl halide, ˜1.4 equiv. in 15 ml of dry DMF was added drop wise at 0° C. followed by addition of ˜0.8 g of potassium carbonate to make pH 8. Reaction was stirred at room temperature for 4 hours and then poured into 75 ml of ice water. Mixture was then transferred to the 250 ml separatory funnel and extracted with 50 ml of ethyl ether for three times. Combined ether solution was then dried over magnesium sulfate, filtered and evaporated to get crude product.

Column chromatography of the crude product resulted in pure product in 79-92% yield.

### Ethyl 6-ethoxy-3-(3-(ethoxycarbonyl)benzyl)-1-((6-ethylbenzo[d][1,3]dioxol-5-yl)methyl)-4-oxo-1,4-dihydroquinoline-2-carboxylate, 1b (R, R′, R″═C₂H₄)

Product 1a, 0.45 g (0.808 mmol) was reacted with iodoethane, 0.164 g (1.051 mmol) in presence of 0.045 g of 60% W/W sodium hydride and 0.8 g of potassium carbonate for 4 hours. Column chromatography (60% Hexane: 40% EtOAc) of the crude product resulted in 0.36 g (79%) of the pure product, 1b.

m.p. 124-126° C., TLC (60% Hexane: 40% EtOAc) R_{f}=0.434.

MS: m/z 585 (M⁺), 528 (M-C₂H₅)⁺, 527 (M-CH₂O—), 512 (M-C₂H₅O)⁺, 484 (M—COOC₂H₅)⁺, 408 (C₂₃H₂₂NO₆), 394 [(M-163)⁺, (M-C₁₀H₁₁O₂)^{+],} 366 (394-CO)⁺, 320 [(394-HCOOC₂H₅)⁺, C₁₉H₁₄NO₄], 248 (C₁₆H₁₀NO₂), 220 [(248-CO)⁺, C₁₅H₁₀O], 163 (base peak, +CH₂C₆H₄COOC₂H₅, C₁₀H₁₁O₂), 133 [(163-CH₂O)⁺, C₉H₉O], 105 [(133-CO) or 133-C₂H₄)⁺, C₇H₅O], 91 C₇H₇⁺, 79 C₆H₇⁺, 77 C₆H₅⁺, 55 C₄H₇⁺.

IR: 3019 cm⁻¹ (alkyl), 1732 cm⁻¹ (α β unsaturated ester, C═O), 1709 cm⁻¹ (aromatic ester, C═O), 1597 cm⁻¹ (quinol-4-one, C═O).

¹H-NMR (CDCl₃, δ ppm): a: 1.05, t, 3H, J_{ad}=6.5 Hz; b: 1.27, t, 3H, J_{bf}=6.7 Hz; c: 1.36, t, 3H, J_{cg}=6.8 Hz; A: 1.42, t, 3H, J_{AB}=6.8 Hz; d: 2.65, q, 2H, J_{da}=7.4 Hz; e: 3.99, s, 2H; B: 4.13, q, 2H, J_{BA}=6.8 Hz; f: 4.24, q, 2H, J_{fb}=6.9 Hz; h: 5.19, s, 2H; i: 5.86, s, 2H; j: 6.21, s, 1H; k: 6.74, s, 1H; l: 7.08, d, 1H, Jₗₘ=9.0 Hz; m: 7.15, dd, 1H, Jₘₗ=9.4 Hz; n: 7.31, t, 1H, J=7.7 Hz; o: 7.60, d, 1H, Jₒₙ=7.3 Hz;

p: 7.83, s, 1H; q: 7.84, d, 1H, J_{qn}=8.8 Hz; r: 7.99, s, 1H.

¹³C-NMR (CDCl₃, δ ppm): C₁: 13.19, C₂: 14.54, C₃: 14.76, C_{I}: 14.90, C₄: 25.61, C₅: 32.51, C₆: 51.33, C₇: 60.94, C₈: 62.91, C_{II}: 64.15, C₉: 1.25, C₁₀: 106.5, C₁₁: 106.72, C₁₂: 109.05, C₁₃: 117.82, C₁₄: 118.23, C₁₅: 124.20, C₁₆: 125.62, C₁₇: 127.44, C₁₈: 127.50, C₁₉: 128.44, C₂₀: 129.44, C₂₁: 130.45, C₂₂: 133.59, C₂₃: 134.69, C₂₄: 140.62, C₂₅: 144.16, C₂₆: 146.54, C₂₇: 147.46, C₂₈: 155.99, C₂₉: 163.99, C₃₀: 167.01, C₃₁: 176.72.

### Ethyl 3-(3-(ethoxycarbonyl)benzyl)-1-((6-ethylbenzo[d][1,3]dioxol-5-yl)methyl)-4-oxo-6-propoxy-1,4-dihydroquinoline-2-carboxylate, 1c (R=n-(C₃H₇, R′═C₂H₅, R″═H)

Product 13, 3.5 g (6.283 mmol) was reacted with 1-iodopropane, 1.6 g (8.95 mmol) in presence of 0.35 g of 60% W/W sodium hydride and 4.5 g of potassium carbonate for 4 hours. Column chromatography (60% Hexane: 40% EtOAc) of the crude product resulted in 0.36 g (87.7%) of the pure product, 14c.

m.p. 134-135° C., TLC (50% Hexane: 50% EtOAc) R_{f}=0.653.

MS: m/z 599 (M⁺), 571 [(M-C₂H₄)⁺ or (M-CO)⁺], 570 (M-C₂H₅)⁺, 554 [(M-45), (M-C₂H₅O)⁺, 526 [(M-73), (M—COOC₂H₅)⁺], 436 [(M-163), (M-C₁₀H₁₁O₂)⁺, 408 (C₂₃H₂₂NO₆), 362 (436-HCOOC₂H₅)⁺, 248 (C₁₆H₁₀NO₂), 220 [(248-CO)⁺, C₁₅H₁₀NO], 163 (base peak, +CH₂C₆H₄COOC₂H₅, C₁₀H₁₁O₂), 133 [(163-CH₂O)⁺, C₉H₉O], 105 [(133-CO) or 133-C₂H₄)⁺, C₇H₅O], 91 C₇H₇⁺, 79 C₆H₇⁺, 77 C₆H₅⁺, 55 C₄H₇⁺.

IR: 2965 cm⁻¹ (alkyl), 1738 cm⁻¹ (α β unsaturated ester, C═O), 1714 cm⁻¹ (aromatic ester, C═O), 1597 cm⁻¹ (quinol-4-one, C═O).

¹H-NMR (CDCl₃, δ ppm): A: 1.02, t, 3H, J_{AB}=7.1 Hz; a: 1.05, t, 3H, J_{ad}=7.1 Hz; b: 1.27, t, 3H, J_{bf}=6.8 Hz; c: 1.36, t, 3H, J_{cg}=7.1 Hz; B: 1.81, sextet, 2H, J=6.9 Hz; d: 2.64, q, 2H, J_{da}=7.4 Hz; e: 3.99, s, 2H; C, 4.02, t, 2H, J_{CB}=6.5 Hz; f: 4.24, q, 2H, J_{fb}=7.1 Hz; g: 4.34, q, 2H, J_{gc}=7.1 Hz; h: 5.20, s, 2H; i: 5.86, s, 2H; j: 6.21, s, 1H; k: 6.74, s, 1H; l: 7.08, d, 1H, Jₗₘ=9.2 Hz; m: 7.15, dd, 1H, Jₘₗ=9.3 Hz; n: 7.32, t, 1H, J=7.6 Hz; o: 7.60, d, 1H, J_{qn}=7.3 Hz; p: 7.83, s, 1H; q: 7.84, d, 1H, J_{qn}=7.5 Hz; r: 7.99, s, 1H.

¹³C-NMR (CDCl₃, δ ppm): C₁: 10.65, C₁: 13.68, C₂: 14.53, C₃: 14.75, C_{II}: 22.59, C₄: 25.60, C₅: 32.50, C₆: 51.32, C₇: 60.94, C₈: 62.90, C_{III}: 70.19, C₉: 101.23, C₁₀: 106.48, C₁₁: 106.75, C₁₂: 109.03, C₁₃: 117.79, C₁₄: 118.19, C₁₅: 124.17, C₁₆: 125.61, C₁₇: 127.42, C₁₈: 127.48, C₁₉: 128.44, C₂₀: 129.87, C₂₁: 130.43, C₂₂: 133.59, C₂₃: 134.63, C₂₄: 140.61, C₂₅: 144.13, C₂₆: 146.52, C₂₇: 147.44, C₂₈: 155.99, C₂₉: 163.98, C₃₀: 167.01, C₃₁: 176.71.

### Ethyl 3-(3-(ethoxycarbonyl)benzyl)-1-((6-ethylbenzo[d][1,3]dioxol-5-yl)methyl)-6-isopropoxy-4-oxo-1,4-dihydroquinoline-2-carboxylate, 1d (R=i-C₃H₇, R′═C₂H₅, R″═C₂H₂)

Product 1a, 0.45 g (0.808 mmol) was reacted with 2-iodopropane, 0.164 g (1.051 mmol) in presence of 0.045 g of 60% W/W sodium hydride and 0.8 g of potassium carbonate for 4 hours. Column chromatography (60% Hexane: 40% EtOAc) of the crude product resulted in 0.36 g (74.4%) of the pure product, 1d.

m.p. 124-126° C., TLC (60% Hexane: 40% EtOAc) R_{f}=0.626.

MS: m/z 599 (M⁺), 570 (M-C₂H₅)⁺, 554 [(M-45), (M-C₂H₅O)⁺, 526 [(M-73), (M-COOC₂H₅)⁺], 436 [(M-163), (M-C₁₀H₁₁O₂)⁺], 408 (C₂₃H₂₂NO₆), 362 (436-HCOOC₂H₅)⁺, 320 [(362-C₃H₆)⁺, (C₁₉H₁₄NO₄)], 248 (C₁₆H₁₀NO₂), 220 [(248-CO)⁺, C₁₅H₁₀NO], 163 (base peak, +CH₂C₆H₄COOC₂H₅, C₁₀H₁₁O₂), 133 [(163-CH₂O)⁺, C₉H₉O], 105 [(133-CO) or 133-C₂H₄)⁺, C₇H₅O], 91 C₇H₇⁺, 79 C₆H₇⁺, 77 C₆H₅⁺, 55 C₄H₇⁺.

IR: 3019 cm⁻¹ (alkyl), 1731 cm⁻¹ (α β unsaturated ester, C═O), 1713 cm⁻¹ (aromatic ester, C═O), 1598 cm⁻¹ (quinol-4-one, C═O).

¹H-NMR (CDCl₃, δ ppm): a: 1.05, t, 3H, J_{ad}=7.1 Hz; b: 1.27, t, 3H, J_{bf}=7.5 Hz; A: 1.34, d, 6H, J_{AB}=6.0 Hz; c: 1.36, t, 3H, J_{cg}=7.2 Hz; d: 2.64, q, 2H, J_{da}=7.5 Hz; e: 3.99, s, 2H; f: 4.24, q, 2H, J_{fb}=7.5 Hz; g: 4.34, q, 2H, J_{gc}=7.1 Hz; B: 4.70, septet, 1H, J_{BA}=6.0 Hz; h: 5.19, s, 2H; i: 5.86, s, 2H; j: 6.23, s, 1H; k: 6.74, s, 1H; l: 7.07, d, 1H, Jₗₘ=9.3 Hz; m: 7.11, dd, 1H, Jₘₗ=9.3 Hz; n: 7.32, t, 1H, J=7.7 Hz; o: 7.60, d, 1H, Jₒₙ=7.7 Hz; p: 7.83, s, 1H; q: 7.85, d, 1H, J_{qn}=6.6 Hz; r: 7.99, s, 1H.

¹³C-NMR (CDCl₃, δ ppm): C₁: 13.68, C₂: 14.54, C₃: 14.75, C_{I}: 22.10, C₄: 25.60, C₅: 32.52, C₆: 51.31, C₇: 60.94, C₈: 62.90, C_{II}: 70.51, C₉: 101.24, C₁₀: 106.53, C₁₁: 107.98, C₁₂: 109.04, C₁₃: 117.74, C₁₄: 118.29, C₁₅: 125.06, C₁₆: 125.65, C₁₇: 127.46, C₁₈: 127.49, C₁₉: 128.44, C₂₀: 129.87, C₂₁: 130.46, C₂₂: 133.59, C₂₃: 134.54, C₂₄: 140.65, C₂₅: 144.15, C₂₆: 146.54, C₂₇: 147.45, C₂₈: 155.97, C₂₉: 164.0, C₃₀: 167.02, C₃₁: 176.69.

### Ethyl 6-(allyloxy)-3-(3-(ethoxycarbonyl)benzyl)-1-((6-ethylbenzo[d][1,3]dioxol-5-yl)methyl)-4-oxo-1,4-dihydroquinoline-2-carboxylate, 1e, (R═H₂C═CH—, R′═C₂H₅, R″═C₂H₅H)

Product 1a, 0.40 g (0.718 mmol) was reacted with allyliodide, 0.181 g (1.081 mmol) in presence of 0.045 g of 60% W/W sodium hydride and 0.8 g of potassium carbonate for 4 hours. Column chromatography (60% Hexane: 40% EtOAc) of the crude product resulted in 0.36 g (90.0%) of the pure product, 1e.

m.p. 128-131° C., TLC (60% Hexane: 40% EtOAc) R_{f}=0.645.

MS: m/z 597 (M⁺), 568 (M-C₂H₅)⁺, 552 [(M-45), (M-C₂H₅O′)⁺, 524 [(M-73), (M-COOC₂H₅)⁺], 434 [(M-163), (M-C₁₀H₁₁O₂)⁺], 408 (C₂₃H₂₂NO₆), 388 (434-C₂H₅OH), 360 (434-HCOOC₂H₅)⁺, 288 (C₁₉H₁₄NO₂), 248 (C₁₆H₁₀O₂), 163 (base peak, C₁₀H₁₁O₂, +CH₂C₆H₄COOC₂H₅), 133 [(163-CH₂O)⁺, C₉H₉O], 105 [(133-CO) or 133-C₂H₄)⁺, C₇H₅O], 91 C₇H₇⁺, 79 C₆H₇⁺, 77 C₆H₅⁺, 55 C₄H₇⁺.

IR: 3018 cm⁻¹ (alkyl), 1735 cm⁻¹ (α β unsaturated ester, C═O), 1711 cm⁻¹ (aromatic ester, C═O), 1597 cm⁻¹ (quinol-4-one, C═O).

¹H-NMR (CDCl₃, δ ppm): a: 1.05, t, 3H, J_{ad}=7.1 Hz; b: 1.27, t, 3H, J_{bf}=7.5 Hz; c: 1.36, t, 3H, J_{cg}=7.2 Hz; d: 2.64, q, 2H, J_{da}=7.5 Hz; e: 3.99, s, 2H; f: 4.24, q, 2H, J_{fb}=7.5 Hz; g: 4.34, q, 2H, J_{gc}=7.1 Hz; A: 4.62, d, 2H, J_{AD}=3.8 Hz; h: 5.19, s, 2H; B: 5.29, d, 1H, J_{BD}=17.3 Hz; C, 5.42, d, 1H, J_{CD}=17.3 Hz; i: 5.86, s, 2H; D: 6.05, octet, 1H, J=5.3 Hz; j: 6.22, s, 1H; k: 6.74, s, 1H; l: 7.09, d, 1H, Jₗₘ=9.3 Hz; m: 7.19, dd, 1H, Jₘₗ=9.3 Hz; n: 7.32, t, 1H, J=7.6 Hz; o: 7.60, d, 1H, Jₒₙ=7.3 Hz; p: 7.84, s, 1H; q: 7.85, d, 1H, J_{qn}=6.6 Hz; r: 8.00, s, 1H.

¹³C-NMR (CDCl₃, δ ppm): C₁: 13.66, C₂: 14.52, C₃: 14.74, C₄: 25.58, C₅: 32.47, C₆: 51.34, C₇: 60.93, C₈: 62.91, C_{I}: 69.32, C₉: 101.23, C₁₀: 106.44, C₁₁: 107.12, C₁₂: 109.03, C₁₃: 117.84, C₁₄: 118.30, C_{II}: 118.32, C₁₅: 124.18, C₁₆: 125.52, C₁₇: 127.34, C₁₈: 127.48, C₁₉: 128.42, C₂₀: 129.83, C₂₁: 130.41, C_{III}: 132.86, C₂₂: 133.55, C₂₃: 133.59, C₂₄: 134.84, C₂₅: 140.54, C₂₆: 144.21, C₂₇: 146.51, C₂₈: 147.44, C₂₉: 155.55, C₃₀: 163.92, C₃₁: 166.98, C₃₂: 176.65.879

### Ethyl 6-butoxy-3-(3-(ethoxycarbonyl)benzyl)-1-((6-ethylbenzo[d][1,3]dioxol-5-yl)methyl)-4-oxo-1,4-dihydroquinoline-2-carboxylate, 1 g: (R=n-C₄H₉, R′═C₂H₅, R″═C₂H₅)

Product 1a, 0.40 g (0.718 mmol) was reacted with 1-iodobutane, 0.182 g (1.081 mmol) in presence of 0.045 g of 60% W/W sodium hydride and 0.8 g of potassium carbonate for 4 hours. Column chromatography (60% Hexane: 40% EtOAc) of the crude product resulted in 0.36 g (90.0%) of the pure product, 1 g.

m.p. 147-150° C., TLC (60% Hexane: 40% EtOAc) R_{f}=0.632.

MS: m/z 613 (M⁺), 568 [(M-45), (M-C₂H₅O)⁺], 540 [(M-73), (M-COOC₂H₅)⁺], 450 [(M-163), (M-C₁₀H₁₁O₂)⁺], 422 [(450-28), (M-CO)⁺, (C₂₃H₂₂NO₆)], 376 (436-HCOOC₂H₅)⁺, 320 [(362-C₃H₆)⁺, (C₁₉H₁₄NO₄)], 248 (C₁₆H₁₀NO₂), 220 [(248-CO)⁺, C₁₅H₁₀O], 163 (base peak, +CH₂C₆H₄COOC₂H₅, C₁₀H₁₁O₂), 133 [(163-CH₂O)⁺, C₉H₉O], 105 [(133-CO) or 133-C₂H₄)⁺, C₇H₅O], 91 C₇H₇⁺, 79 C₆H₇⁺, 77 C₆H₅⁺, 55 C₄H₇⁺.

IR: 3019 cm⁻¹ (alkyl), 1732 cm⁻¹ (α β unsaturated ester, C═O), 1711 cm⁻¹ (aromatic ester, C═O), 1597 cm⁻¹ (quinol-4-one, C═O).

¹H-NMR (CDCl₃, δ ppm): A: 0.96, t, 3H, J_{AB}=7.4 Hz; a: 1.05, t, 3H, J_{ad}=7.1 Hz; b: 1.27, t, 3H, J_{bf}=7.5 Hz; B: 1.34, sextet, 2H, J=7.5 Hz; c: 1.36, t, 3H, J_{cg}=7.2 Hz; C, 1.34, quintet, 2H, J=7.7 Hz; d: 2.64, q, 2H, J_{da}=7.5 Hz; e: 3.99, s, 2H; D: 4.06, t, 2H, J_{DC}=6.0 Hz; f: 4.23, q, 2H, J_{fb}=7.2 Hz; g: 4.34, q, 2H, J_{gc}=7.1 Hz; h: 5.19, s, 2H; i: 5.86, s, 2H; j: 6.21, s, 1H; k: 6.74, s, 1H; l: 7.07, d, 1H, J=9.4 Hz; m: 7.11, dd, 1H, Jₘₗ=9.3 Hz; n: 7.32, t, 1H, J=7.7 Hz; o: 7.60, d, 1H, Jₒₙ=7.7 Hz; p: 7.83, s, 1H; q: 7.84, d, 1H, J_{qn}=6.3 Hz; r: 7.99, s, 1H.

¹³C-NMR (CDCl₃, δ ppm): C₁: 13.68, C_{I}: 13.98, C₂: 14.54, C₃: 14.76, C_{II}: 19.40, C₄: 25.61, C_{III}: 31.32, C₅: 32.52, C₆: 51.33, C₇: 60.94, C₈: 62.90, C_{IV}: 68.42, C₉: 101.24, C₁₀: 106.51, C₁₁: 106.76, C₁₂: 109.04, C₁₃: 117.82, C₁₄: 118.19, C₁₅: 125.06, C₁₆: 125.64, C₁₇: 127.45, C₁₈: 127.49, C₁₉: 128.44, C₂₀: 129.88, C₂₁: 130.45, C₂₂: 133.59, C₂₃: 134.65, C₂₄: 140.64, C₂₅: 144.14, C₂₆: 146.55, C₂₇: 147.46, C₂₈: 155.21, C₂₉: 163.99, C₃₀: 167.01, C₃₁: 176.72.

### Ethyl 3-(3-(ethoxycarbonyl)benzyl)-1-((6-ethylbenzo[d][1,3]dioxol-5-yl)methyl)-6-isobutoxy-4-oxo-1,4-dihydroquinoline-2-carboxylate, 1 h: (R=i-C₄H₉, R′═C₂H₅, R″═C₂H₅)

Product 1a, 0.40 g (0.718 mmol) was reacted with 2-methyl-1-iodopropane, 0.182 g (1.081 mmol) in presence of 0.045 g of 60% W/W sodium hydride and 0.8 g of potassium carbonate for 4 hours. Column chromatography (60% Hexane: 40% EtOAc) of the crude product resulted in 0.33 g (81.3%) of the pure product, 1 h.

m.p. 129-132° C., TLC (50% Hexane: 50% EtOAc) R_{f}=0.722.

MS: m/z 613 (M⁺), 568 [(M-45), (M-C₂H₅O)⁺], 540 [(M-73), (M-COOC₂H₅)⁺], 450 [(M-163), (M-C₁₀H₁₁O₂)⁺], 422 [(450-28), (M-CO)⁺, (C₂₃H₂₂NO₆)], 376 (436-HCOOC₂H₅)⁺, 320 [(362-C₃H₆)⁺, (C₁₉H₁₄NO₄)], 248 (C₁₆H₁₀NO₂), 220 [(248-CO)⁺, C₁₅H₁₀NO], 163 (base peak, +CH₂C₆H₄COOC₂H₅, C₁₀H₁₁O₂), 133 [(163-CH₂O)⁺, C₉H₉O], 105 [(133-CO) or 133-C₂H₄)⁺, C₇H₅O], 91 C₇H₇⁺, 79 C₆H₇⁺, 77 C₆H₅⁺, 55 C₄H₇⁺.

IR: 3019 and 2978 cm⁻¹ (alkyl), 1731 cm⁻¹ (α β unsaturated ester, C═O), 1717 cm⁻¹ (aromatic ester, C═O), 1596 cm⁻¹ (quinol-4-one, C═O).

¹H-NMR (CDCl₃, δ ppm): A: 0.95, t, 3H, J_{AB}=7.4 Hz; a: 1.05, t, 3H, J_{ad}=7.1 Hz; b: 1.27, t, 3H, J_{bf}=7.4 Hz; B: 1.30, d, 3H, J_{BE}=6.1 Hz; c: 1.36, t, 3H, J_{cg}=7.2 Hz; C, 1.63, octet, 1H, J=6.8 Hz; D: 1.34, octet, 1H, J=7.1 Hz; d: 2.65, q, 2H, J_{da}=7.4 Hz; e: 3.99, s, 2H; f: 4.24, q, 2H, J_{fb}=7.1 Hz; g: 4.34, q, 2H, J_{gc}=6.9 Hz; E: 4.46, sextet, 1H, J=6.0 Hz; h: 5.19, s, 2H; i: 5.86, s, 2H; j: 6.24, s, 1H; k: 6.74, s, 1H; l: 7.07, d, 1H, Jₗₘ=9.3 Hz; m: 7.11, dd, 1H, Jₘₗ=9.3 Hz; n: 7.32, t, 1H, J=7.7 Hz; o: 7.60, d, 1H, Jₒₙ=7.4 Hz; p: 7.83, s, 1H; q: 7.84, d, 1H, J_{qn}=6.5 Hz; r: 7.99, s, 1H.

¹³C-NMR (CDCl₃, δ ppm): C_{I}: 9.96, C₁: 13.69, C₂: 14.54, C₃: 14.76, C_{II}: 19.28, C₄: 25.60, C_{III}: 29.25, C₅: 32.53, C₆: 51.31, C₇: 60.94, C₈: 62.90, C_{IV}: 75.62, C₉: 101.24, C₁₀: 106.53, C₁₁: 107.94, C₁₂: 109.03, C₁₃: 117.69, C₁₄: 118.29, C₁₅: 125.05, C₁₆: 125.64, C₁₇: 127.44, C₁₈: 127.48, C₁₉: 128.44, C₂₀: 129.88, C₂₁: 130.43, C₂₂: 133.58, C₂₃: 134.48, C₂₄: 140.64, C₂₅: 144.13, C₂₆: 146.52, C₂₇: 147.43, C₂₈: 155.30, C₂₉: 164.00, C₃₀: 167.01, C₃₁: 176.69.

### Ethyl 6-sec-butoxy-3-(3-(ethoxycarbonyl)benzyl)-1-((6-ethylbenzo[d][1,3]dioxol-5-yl)methyl)-4-oxo-1,4-dihydroquinoline-2-carboxylate, 1i: (R=sec-C₄H₉, R′═C₂H₅, R″═C₂H₅)

Product 1a, 0.40 g (0.718 mmol) was reacted with 2-iodobutane, 0.182 g (1.081 mmol) in presence of 0.045 g of 60% W/W sodium hydride and 0.8 g of potassium carbonate for 4 hours. Column chromatography (60% Hexane: 40% EtOAc) of the crude product resulted in 0.37 g (92.5%) of the pure product, 1i.

m.p. 129-132° C., TLC (60% Hexane: 40% EtOAc) R_{f}=0.600.

MS: m/z 613 (M⁺), 568 [(M-45), (M-C₂H₅O)⁺], 540 [(M-73), (M-COOC₂H₅)⁺], 450 [(M-163), (M-C₁₀H₁₁O₂)⁺], 422 [(450-28), (M-CO)⁺, (C₂₃H₂₂NO₆)], 376 (436-HCOOC₂H₅)⁺, 320 [(362-C₃H₆)⁺, (C₁₉H₁₄NO₄)], 248 (C₁₆H₁₀NO₂), 220 [(248-CO)⁺, C₁₅H₁₀O], 163 (base peak, +CH₂C₆H₄COOC₂H₅, C₁₀H₁₁O₂), 133 [(163-CH₂O)⁺, C₉H₉O], 105 [(133-CO) or 133-C₂H₄)⁺, C₇H₅O], 91 C₇H₇⁺, 79 C₆H₇⁺, 77 C₆H₅⁺, 55 C₄H₇⁺.

IR: 3019 and 2966 cm⁻¹ (alkyl), 1733 cm⁻¹ (α β unsaturated ester, C═O), 1718 cm⁻¹ (aromatic ester, C═O), 1597 cm⁻¹ (quinol-4-one, C═O).

¹H-NMR (CDCl₃, δ ppm): A: 1.00, d, 6H, J_{AB}=6.8 Hz; a: 1.05, t, 3H, J_{ad}=7.1 Hz; b: 1.27, t, 3H, J_{bf}=7.5 Hz; c: 1.36, t, 3H, J_{cg}=7.1 Hz; B: 2.11, nonet, 1H, J=6.7 Hz; d: 2.65, q, 2H, J_{da}=7.5 Hz; C, 3.82, d, 2H, J=6.6 Hz; e: 3.99, s, 2H; f: 4.24, q, 2H, J_{fb}=7.1 Hz; g: 4.34, q, 2H, J_{gc}=7.1 Hz; h: 5.19, s, 2H; i: 5.85, s, 2H; j: 6.21, s, 1H; k: 6.74, s, 1H; l: 7.08, d, 1H, Jₗₘ=9.4 Hz; m: 7.11, dd, 1H, Jₘₗ=9.3 Hz; n: 7.31, t, 1H, J=7.7 Hz; o: 7.60, d, 1H, J_{qn}=7.6 Hz; p: 7.82, s, 1H; q: 7.84, d, 1H, J_{qn}=8.9 Hz; r: 7.99, s, 1H.

¹³C-NMR (CDCl₃, δ ppm): C₁: 13.70, C₂: 14.55, C₃: 14.78, C_{I}: 19.41, C₄: 25.60, C_{II}: 28.31, C₅: 32.53, C₆: 51.33, C₇: 60.96, C₈: 62.92, C_{III}: 75.11, C₉: 101.25, C₁₀: 106.52, C₁₁: 106.82, C₁₂: 109.05, C₁₃: 117.83, C₁₄: 118.19, C₁₅: 124.19, C₁₆: 125.65, C₁₇: 127.45, C₁₈: 127.50, C₁₉: 128.45, C₂₀: 129.90, C₂₁: 130.45, C₂₂: 133.62, C₂₃: 134.63, C₂₄: 140.65, C₂₅: 144.14, C₂₆: 146.56, C₂₇: 147.47, C₂₈: 155.32, C₂₉: 164.01, C₃₀: 167.04, C₃₁: 176.74.

### Ethyl 6-(cyclopropylmethoxy)-3-(3-(ethoxycarbonyl)benzyl)-1-((6-ethylbenzo[d][1,3]dioxol-5-yl)methyl)-4-oxo-1,4-dihydroquinoline-2-carboxylate, 1j: (R=cyclo-C₃H₅CH₂, R′═C₂H₅, R″═C₂H₅)

Product 1a, 0.40 g (0.718 mmol) was reacted with 2-iodobutane, 0.182 g (1.081 mmol) in presence of 0.045 g of 60% W/W sodium hydride and 0.8 g of potassium carbonate for 4 hours. Column chromatography (60% Hexane: 40% EtOAc) of the crude product resulted in 0.37 g (81%) of the pure product, 1j.

m.p. 129-132° C., TLC (60% Hexane: 40% EtOAc) R_{f}=0.54.

MS: m/z 613 (M⁺), 568 [(M-45), (M-C₂H₅O)⁺], 540 [(M-73), (M-COOC₂H₅)⁺], 450 [(M-163), (M-C₁₀H₁₁O₂)⁺], 422 [(450-28), (M-CO)⁺, (C₂₃H₂₂NO₆)], 376 (436-HCOOC₂H₅)⁺, 320 [(362-C₃H₆)⁺, (C₁₉H₁₄NO₄)], 248 (C₁₆H₂₂NO₂), 220 [(248-CO)⁺, C₁₅H₁₀NO], 163 (base peak, +CH₂C₆H₄COOC₂H₅, C₁₀H₁₁O₂), 133 [(163-CH₂O)⁺, C₉H₉O], 105 [(133-CO) or 133-C₂H₄)⁺, C₇H₅O], 91 C₇H₇⁺, 79 C₆H₇⁺, 77 C₆H₅⁺, 55 C₄H₇⁺.

IR: 3019 and 2966 cm⁻¹ (alkyl), 1733 cm⁻¹ (α β unsaturated ester, C═O), 1718 cm⁻¹ (aromatic ester, C═O), 1597 cm⁻¹ (quinol-4-one, C═O).

¹H-NMR (CDCl₃, δ ppm): A: 0.33, d, 2H, J_{AB}=4.4 Hz; B: 0.56, d, 2H, J_{BA}=7.8 Hz; a: 1.05, t, 3H, J_{ad}=7.1 Hz; C, 1.20, t, 1H, J=7.4 Hz; b: 1.27, t, 3H, J_{bf}=7.5 Hz; c: 1.36, t, 3H, J_{cg}=7.1 Hz; B: 2.11, nonet, 1H, J=6.7 Hz; d: 2.65, q, 2H, J_{da}=7.5 Hz; C, 3.82, d, 2H, J=6.6 Hz; e: 3.99, s, 2H; f: 4.24, q, 2H, J_{fb}=7.1 Hz; g: 4.34, q, 2H, J_{gc}=7.1 Hz; h: 5.19, s, 2H; i: 5.85, s, 2H; j: 6.21, s, 1H; k: 6.74, s, 1H; l: 7.08, d, 1H, Jₗₘ=9.4 Hz; m: 7.11, dd, 1H, Jₘₗ=9.3 Hz; n: 7.31, t, 1H, J=7.7 Hz; o: 7.60, d, 1H, Jₒₙ=7.6 Hz; p: 7.82, s, 1H; q: 7.84, d, 1H, J_{qn}=8.9 Hz; r: 7.99, s, 1H.

¹³C-NMR (CDCl₃, δ ppm): C_{I}: 3.12, C_{II}: 10.00, C₁: 13.70; C₂: 14.55, C₃: 14.78, C₄: 25.60, C₅: 32.53, C₆: 51.33, C₇: 60.96, C₈: 62.92, C_{III}: 72.40, C_{III}: 72.40, C₉: 101.25, C₁₀: 106.52, C₁₁: 106.82, C₁₂: 109.05, C₁₃: 117.83, C₁₄: 118.19, C₁₅: 124.19, C₁₆: 125.65, C₁₇: 127.45, C₁₈: 127.50, C₁₉: 128.45, C₂₀: 129.90, C₂₁: 130.45, C₂₂: 133.62, C₂₃: 134.63, C₂₄: 140.65, C₂₅: 144.14, C₂₆: 146.56, C₂₇: 147.47, C₂₈: 155.32, C₂₉: 164.01, C₃₀: 167.04, C₃₁: 176.74.

### Ethyl 3-(3-(ethoxycarbonyl)benzyl)-1-((6-ethylbenzo[d][1,3]dioxol-5-yl)methyl)-4-oxo-6-(pentyloxy)-1,4-dihydroquinoline-2-carboxylate, 1k: (R=n-C₅H₁₁, R′═C₂H₅, R″═C₂H₅)

Product 1k, 0.40 g (0.718 mmol) was reacted with 1-iodopentane, 0.214 g (1.081 mmol) in presence of 0.055 g of 60% W/W sodium hydride and 0.8 g of potassium carbonate for 4 hours. Column chromatography (60% Hexane: 40% EtOAc) of the crude product resulted in 0.38 g (84.4%) of the pure product, 1k.

m.p. 98-99° C., TLC (70% Hexane: 30% EtOAc) R_{f}=0.555.

MS: m/z 627 (M⁺), 582 [(M-45), (M-C₂H₅O)⁺], 554 [(M-73), (M-COOC₂H₅)⁺], 464 [(M-163), (M-C₁₀H₁₁O₂)⁺], 436 [(464-28), (M-CO)⁺], 390 (464-HCOOC₂H₅)⁺320-[(362-C₅H₁₀)⁺C₁₉H₁₄NO₄)], 376 (436-HCOOC₂H₅)⁺, 320 [(362-C₃H₆)⁺, (C₁₉H₁₄NO₄)], 248 (C₁₆H₁₀NO₂), 220 [(248-CO)⁺, C₁₅H₁₀NO], 163 (base peak, +CH₂C₆H₄COOC₂H₅, C₁₀H₁₁O₂), 133 [(163-CH₂O)⁺, C₉H₉O], 105 [(133-CO) or 133-C₂H₄)⁺, C₇H₅O], 91 C₇H₇⁺, 79 C₆H₇⁺, 77 C₆H₅⁺, 55 C₄H₇⁺.

IR: 3019 cm⁻¹ (alkyl), 1732 cm⁻¹ (α β unsaturated ester, C═O), 1713 cm⁻¹ (aromatic ester, C═O), 1597 cm⁻¹ (quinol-4-one, C═O).

¹H-NMR (CDCl₃, δ ppm): A: 0.91, t, 3H, J_{AB}=6.0 Hz; a: 1.05, t, 3H, J_{ad}=6.0 Hz; b: 1.27, t, 3H, J_{bf}=6.2 Hz; c: 1.36, t, 3H, J_{cg}=7.2 Hz; B: 1.38, m, 4H, J=5.5 Hz; C, 1.79, quintet, 2H, J=6.7 Hz; d: 2.64, q, 2H, J_{da}=6.7 Hz; e: 3.99, s, 2H; D: 4.05, t, 2H, J_{DC}=5.5 Hz; f: 4.23, q, 2H, J_{fb}=6.1 Hz; g: 4.34, q, 2H, J_{gc}=6.0 Hz; h: 5.19, s, 2H; i: 5.86, s, 2H; j: 6.21, s, 1H; k: 6.74, s, 1H; l: 7.07, d, 1H, Jₗₘ=8.8 Hz; m: 7.15, dd, 1H, Jₘₗ=8.5 Hz; n: 7.31, t, 1H, J=6.9 Hz; o: 7.60, d, 1H, Jₒₙ=6.6 Hz; p: 7.83, s, 1H; q: 7.84, d, 1H, J_{qn}=6.3 Hz; r: 7.99, s, 1H.

¹³C-NMR (CDCl₃, δ ppm): C₁: 13.68, C_{I}: 14.19, C₂: 14.53, C₃: 14.75, C_{II}: 22.59, C₄: 25.60, C_{III}: 28.32, C_{IV}: 28.96, C₅: 32.51, C₆: 51.32, C₇: 60.94, C₈: 62.91, C_{V}: 68.69, C₉: 101.24, C₁₀: 106.49, C₁₁: 106.71, C₁₂: 109.03, C₁₃: 117.80, C₁₄: 118.20, C₁₅: 124.19, C₁₆: 125.61, C₁₇: 127.43, C₁₈: 127.49, C₁₉: 128.44, C₂₀: 129.87, C₂₁: 130.43, C₂₂: 133.59, C₂₃: 134.63, C₂₄: 140.62, C₂₅: 144.12, C₂₆: 146.52, C₂₇: 147.44, C₂₈: 155.20, C₂₉: 163.61, C₃₀: 167.01, C₃₁: 176.72.

### Ethyl 3-(3-(ethoxycarbonyl)benzyl)-1-((6-ethylbenzo[d][1,3]dioxol-5-yl)methyl)-6-(3-hydroxypropoxy)-4-oxo-1,4-dihydroquinoline-2-carboxylate, 1l: (R═HO—C₃H₆—, R′═C₂H₅, R″═C₂H₅)

Product 1a, 0.40 g (0.718 mmol) was reacted with 1-iodopropan-3-ol, 0.20 g (1.081 mmol) in presence of 0.045 g of 60% W/W sodium hydride and 0.8 g of potassium carbonate for 4 hours. Column chromatography (60% Hexane: 40% EtOAc) of the crude product resulted in 0.36 g (81.8%) of the pure product, 1l.

m.p. 129-132° C., TLC (60% Hexane: 40% EtOAc) R_{f}=0.546.

MS: m/z 615 (M⁺), 597 [(M-18), (—H₂O)⁺], 587 [(M-C₂H₄)⁺ or (M-CO)⁺], 586 (M-C₂H₅)⁺, 570 [(M-45), (M-C₂H₅O)⁺, 532 [(M-73), (M-COOC₂H₅)⁺], 452 [(M-163), (M-C₁₀H₁₁O₂)⁺, 408 (C₂₃H₂₂NO₆), 378 (452-HCOOC₂H₅)⁺, 248 (C₁₆H₁₀NO₂), 220 [(248-CO)⁺, C₁₅H₁₀NO], 163 (base peak, ⁺CH₂C₆H₄COOC₂H₅, C₁₀H₁₁O₂), 133 [(163-CH₂O)⁺, C₉H₉O], 105 [(133-CO) or 133-C₂H₄)⁺, C₇H₅O], 91 C₇H₇⁺, 79 C₆H₇⁺, 77 C₆H₅⁺, 55 C₄H₇⁺.

IR: 3019 cm⁻¹ (alkyl), 1733 cm⁻¹ (α β unsaturated ester, C═O), 1713 cm⁻¹ (aromatic ester, C═O), 1598 cm⁻¹ (quinol-4-one, C═O).

¹H-NMR (CDCl₃, δ ppm): a: 1.05, t, 3H, J_{ad}=6.9 Hz; b: 1.27, t, 3H, J_{bf}=7.1 Hz; c: 1.36, t, 3H, J_{cg}=6.9 Hz; A: 1.94, bs, 1H; B: 2.06, quintet, 2H, J=6.1 Hz; d: 2.64, q, 2H, J_{da}=7.4 Hz; C, 3.84, t, 2H, J=6.5 Hz; e: 3.99, s, 2H; D: 4.22, t, 2H, J_{DB}=5.0 Hz; f: 4.24, q, 2H, J_{fb}=7.0 Hz; g: 4.34, q, 2H, J_{gc}=7.0 Hz; h: 5.20, s, 2H; i: 5.86, s, 2H; j: 6.20, s, 1H; k: 6.74, s, 1H; l: 7.08, d, 1H, Jₗₘ=9.2 Hz; m: 7.14, d, 1H, Jₘₗ=9.3 Hz; n: 7.31, t, 1H, J=7.6 Hz; o: 7.59, d, 1H, Jₒₙ=7.5 Hz; p: 7.83, d, 1H, Jₚₙ=8.0 Hz; q: 7.84, s, 1H; r: 7.99, s, 1H.

¹³C-NMR (CDCl₃, δ ppm): C₁: 13.69, C₂: 14.54, C₃: 14.77, C₄: 25.61, C_{I}: 32.08, C₅: 32.48, C₆: 51.35, C₇: 60.46, C_{II}: 60.96, C₈: 62.90, C_{III}: 66.32, C₉: 101.26, C₁₀: 106.46, C₁₁: 107.00, C₁₂: 109.06, C₁₃: 117.90, C₁₄: 118.32, C₁₅: 124.02, C₁₆: 125.55, C₁₇: 127.40, C₁₈: 127.51, C₁₉: 128.45, C₂₀: 129.87, C₂₁: 130.45, C₂₂: 133.58, C₂₃: 133.62, C₂₄: 134.84, C₂₅: 140.57, C₂₆: 144.26, C₂₇: 146.55, C₂₈: 147.48, C₂₉: 155.82, C₃₀: 163.94, C₃₁: 167.01, C₃₂: 176.70.

### Ethyl 6-(2-ethoxy-2-oxoethoxy)-3-(3-(ethoxycarbonyl)benzyl)-1-((6-ethylbenzo[d][1,3]dioxol-5-yl)methyl)-4-oxo-1,4-dihydroquinoine-2-carboxylate 1m: (R═C₂H₅OC(O)CH₂, R′═C₂H₅, R″═C₂H₅)

Product 1a, 0.40 g (0.718 mmol) was reacted with bromoethylacetate, 0.18 g (1.077 mmol) in presence of 0.055 g of 60% W/W sodium hydride and 0.8 g of potassium carbonate for 4 hours. Column chromatography (50% Hexane: 50% EtOAc) of the crude product resulted in 0.32 g (70.0%) of the pure product, 1m.

m.p. 129-132° C., TLC (50% Hexane: 50% EtOAc) R_{f}=0.660.

IR: 3019 cm⁻¹ (alkyl), 1733 cm⁻¹ (α β unsaturated ester, C═O), 1713 cm⁻¹ (aromatic ester, C═O), 1598 cm⁻¹ (quinol-4-one, C═O).

¹H-NMR (CDCl₃, δ ppm): a: 1.05, t, 3H, J_{ad}=7.0 Hz; b: 1.27, t, 3H, J_{bf}=7.4 Hz; A: 1.30, t, 3H, J_{AB}=7.3 Hz; c: 1.36, t, 3H, J_{cg}=7.1 Hz; d: 2.64, q, 2H, J_{da}=7.4 Hz; e: 3.99, s, 2H; f: 4.24, q, 2H, J_{fb}=7.2 Hz; B: 4.26, q, 2H, J_{BA}=7.1 Hz; g: 4.34, q, 2H, J_{gc}=7.1 Hz; C, 4.71, s, 2H; h: 5.20, s, 2H; i: 5.87, s, 2H; j: 6.20, s, 1H; k: 6.74, s, 1H; l: 7.12, d, 1H, Jₗₘ=9.4 Hz; m: 7.14, d, 1H, Jₘₗ=7.7 Hz; n: 7.32, t, 1H, J=7.7 Hz; o: 7.58, d, 1H, Jₒₙ=7.5 Hz; p: 7.78, s, 1H; q: 7.84, d, 1H, J_{qn}=7.5 Hz; r: 7.99, s, 1H.

¹³C-NMR (CDCl₃, δ ppm): C₁: 13.67, C_{I}: 14.33, C₂: 14.54, C₃: 14.75, C₄: 25.60, C₅: 32.47, C₆: 51.40, C₇: 60.94, C_{II}: 61.67, C₈: 62.90, C_{III}: 65.54, C₉: 101.26, C₁₀: 106.45, C₁₁: 107.04, C₁₂: 109.07, C₁₃: 118.06, C₁₄: 118.61, C₁₅: 124.23, C₁₆: 125.41, C₁₇: 127.22, C₁₈: 127.53, C₁₉: 128.45, C₂₀: 129.83, C₂₁: 130.47, C₂₂: 133.51, C₂₃: 133.60, C₂₄: 135.40, C₂₅: 140.45, C₂₆: 144.40, C₂₇: 146.56, C₂₈: 147.51, C₂₉: 154.80, C₃₀: 163.85, C₃₁: 166.97, C_{IV}: 168.53, C₃₂: 176.70.

### General Procedure for the Synthesis of 1,3,6-Trisubstituted-2-Carboxylic Acid-Quinol-4-ones

In a solution of adequate 1,3,6-Trisubstituted-2-ethylcarboxylate-quinol-4-ones (1a-m) in an aqueous ethanol (85%), add six equivalents of 6N KOH and reflux overnight. Reaction was monitored by TLC and on completion solvent was evaporated on rotary evaporation. The residue was dissolved in minimum amount of water and solution acidified with equal amount of 1.0 N HCl to get precipitate. Precipitate filtered under vacuum, washed thoroughly with water and allowed to dry. Recrystallization from methanol-water resulted in analytically pure compounds with sharp melting point.

### 3-(3-carboxybenzyl)-1-((6-ethylbenzo[d][1,3]dioxol-5-yl)methyl)-6-hydroxy-4-oxo-1,4-dihydroquinoline-2-carboxylic acid, 1n (R═OH, R′═C₂H₅, R″═H)

Recrystallization from methanol-water (98:2) resulted in 0.36 g (80.0%) of pure product.

m.p.=206-208° C., TLC (70% EtOAc: 30% methanol) R_{f}=0.623.

IR: 3506 cm⁻¹ (carboxylic acid —OH), 3258 cm⁻¹ (phenolic —OH), 1693 cm⁻¹ (α β unsaturated acid, C═O), 1693 cm⁻¹ (aromatic acid, C═O), 1587 cm⁻¹ (quinol-4-one, C═O).

¹H-NMR (DMSO-d₆, δ ppm): a: 1.19, t, 3H, J_{ab}=7.6 Hz; b: 2.69, q, 2H, J_{ba}=7.3 Hz; c: 3.86, s, 2H; d: 5.37, s, 2H; e: 5.89, s, 1H; f: 5.93, s, 1H; g: 6.86, s, 1H; h: 7.14, dd, 1H, Jₕᵢ=9.3 Hz; i: 7.21, d, 1H, Jᵢₕ=9.1 Hz; j: 7.35, t, 1H, J=7.7 Hz; k: 7.52, d, 1H, Jₖⱼ=6.3 Hz; l: 7.53, s, 1H; m: 7.72, d, 1H, Jₘⱼ=7.6 Hz; n: 7.87, s, 1H; A: 9.91 (exchangeable with D₂O), s, 1H; o: 12.83 (exchangeable with D₂O), bs, 2H.

¹³C-NMR (DMSO-d₆, δ ppm): C₁: 14.63, C₂: 24.55, C₃: 32.02, C₄: 50.32, C₅: 100.89, C₆: 105.21, C₇: 108.27, C₈: 108.89, C₉: 114.23, C₁₀: 119.20, C₁₁: 122.59, C₁₂: 126.28, C₁₃: 126.72, C₁₄: 126.80, C₁₅: 128.09, C₁₆: 129.34, C₁₇: 130.46, C₁₈: 132.81, C₁₉: 132.86, C₂₀: 133.96, C₂₁: 140.88, C₂₂: 145.50, C₂₃: 145.99, C₂₄: 146.49, C₂₅: 154.12, C₂₆: 164.98, C₂₇: 167.45, C₂₈: 175.06.

Anal.: Calc. for C₂₈H₂₃NO₈. ½H₂O: C, 65.88; H, 4.70; N, 2.74. Found: C, 65.79; H, 4.66; N, 2.89.

### 3-(3-carboxybenzyl)-6-ethoxy-1-((6-ethylbenzo[d][1,3]dioxol-5-yl)methyl)-4-oxo-1,4-dihydroquinoline-2-carboxylic acid, 1o: (R, R′═C₂H₅, R″═H):

Recrystallization from methanol-water (98:2) resulted in 0.22 g (81.0%) of pure product.

m.p.=213-215° C., TLC (70% EtOAc: 30% methanol) R_{f}=0.662.

IR: 3426 cm⁻¹ (carboxylic acid —OH), 1682 cm⁻¹ (α β unsaturated acid, C═O), 1682 cm⁻¹ (aromatic acid, C═O), 1589 cm⁻¹ (quinol-4-one, C═O).

¹H-NMR (DMSO-d₆, δ ppm): a: 1.20, t, 3H, J_{ab}=7.3 Hz; A: 1.34, t, 3H, J_{AB}=6.7 Hz; b: 2.69, q, 2H, J_{ba}=7.4 Hz; c: 3.88, s, 2H; B: 4.09, q, 2H, J_{BA}=6.8 Hz; d: 5.41, s, 2H; e: 5.89, s, 1H; f: 5.91, s, 1H; g: 6.87, s, 1H; h: 7.14, d, 2H; i: 7.36, t, 1H, J=7.7 Hz; j: 7.54, d, 1H, Jⱼᵢ=7.5 Hz; k: 7.60, s, 1H; l: 7.72, d, 1H, Jₗⱼ=7.4 Hz; m: 7.89, s, 1H; n: 12.84 (exchangeable with D₂O), bs, 2H.

¹³C-NMR (DMSO-d₆, δ ppm): C_{I}: 14.53, C₁: 14.61, C₂: 24.55, C₃: 32.06, C₄: 50.39, C_{II}: 63.44, C₅: 100.89, C₆: 105.15, C₇: 105.82, C₈: 108.92, C₉: 114.85, C₁₀: 119.38, C₁₁: 122.98, C₁₂: 126.16, C₁₃: 126.39, C₁₄: 126.83, C₁₅: 128.09, C₁₆: 129.30, C₁₇: 130.46, C₁₈: 132.91, C₁₉: 133.80, C₂₀: 133.99, C₂₁: 140.73, C₂₂: 145.51, C₂₃: 146.06, C₂₄: 146.52, C₂₅: 154.12, C₂₆: 164.89, C₂₇: 167.44, C₂₈: 175.01.

### 3-(3-carboxybenzyl)-1-((6-ethylbenzo[d][1,3]dioxol-5-yl)methyl)-4-oxo-6-propoxy-1,4-dihydroquinoline-2-carboxylic acid, 1p: (R=n-C₃H₇, R′═C₂H₅, R″═H):

Recrystallization from methanol-water (98:2) resulted in 0.14 g (70.0%) of pure product.

m.p.=162-163° C., TLC (70% EtOAc: 30% methanol) R_{f}=0.675.

IR: 3426 cm⁻¹ (carboxylic acid —OH), 1685 cm⁻¹ (α β unsaturated acid, C═O), 1685 cm⁻¹ (aromatic acid, C═O), 1589 cm⁻¹ (quinol-4-one, C═O).

¹H-NMR (DMSO-d₆, δ ppm): A: 0.97, t, 3H, J_{AB}=7.4 Hz; a: 1.20, t, 3H, J_{ab}=7.5 Hz; B: 1.74, sextet, 2H, J=6.9 Hz; b: 2.69, q, 2H, J_{ba}=7.4 Hz; c: 3.88, s, 2H; C, 3.99, t, 2H, J_{CB}=6.4 Hz; d: 5.41, s, 2H; e: 5.89, s, 1H; f: 5.91, s, 1H; g: 6.87, s, 1H; h: 7.28, d, 2H; i: 7.36, t, 1H, J=7.6 Hz; j: 7.54, d, 1H, Jⱼᵢ=7.5 Hz; k: 7.60, s, 1H; l: 7.73, d, 1H, Jₗⱼ=7.6 Hz; m: 7.89, s, 1H; n: 12.86 (exchangeable with D₂O), bs, 2H.

¹³C-NMR (DMSO-d₆, δ ppm): C_{I}: 10.34, C₁: 14.62, C_{II}: 21.90, C₂: 24.56, C₃: 32.07, C₄: 50.41, C_{II}: 69.32, C₅: 100.90, C₆: 105.17, C₇: 105.89, C₈: 108.92, C₉: 114.83, C₁₀: 119.38, C₁₁: 122.98, C₁₂: 126.18, C₁₃: 126.40, C₁₄: 126.83, C₁₅: 128.09, C₁₆: 129.32, C₁₇: 130.46, C₁₈: 132.92, C₁₉: 133.80, C₂₀: 133.99, C₂₁: 140.75, C₂₂: 145.52, C₂₃: 146.15, C₂₄: 146.52, C₂₅: 155.27, C₂₆: 164.09, C₂₇: 167.45, C₂₈: 175.02.

Anal.: Calc. for C₃₁H₂₉NO₈.½H₂O: C, 67.39; H, 5.52; N, 2.53. Found: C, 67.24; H, 5.34; N, 2.49.

### 3-(3-carboxybenzyl)-1-((6-ethylbenzo[d][1,3]dioxol-5-yl)methyl)-6-isopropoxy-4-oxo-1,4-dihydroquinoline-2-carboxylic acid, 1q: (R=i-C₃H₇, R′═C₂H₅, R″═H):

Recrystallization from methanol-water (98:2) resulted in 0.19 g (79.0%) of pure product.

m.p.=199-200° C., TLC (70% EtOAc: 30% methanol) R_{f}=0.650.

IR: 3409 cm⁻¹ (carboxylic acid —OH), 1687 cm⁻¹ (α β unsaturated acid, C═O), 1687 cm⁻¹ (aromatic acid, C═O), 1589 cm⁻¹ (quinol-4-one, C═O).

¹H-NMR (DMSO-d₆, δ ppm): a: 1.20, t, 3H, J_{ab}=6.8 Hz; A: 1.74, d, 6H, J=5.1 Hz; b: 2.69, q, 2H, J_{ba}=6.8 Hz; c: 3.88, s, 2H; B: 3.99, septet, 1H, J_{BA}=6.1 Hz; d: 5.41, s, 2H; e: 5.90, s, 1H; f: 5.94, s, 1H; g: 6.87, s, 1H; h: 7.27, d, 2H; i: 7.37, t, 1H, J=7.2 Hz; j: 7.54, d, 1H, Jⱼᵢ=7.2 Hz; k: 7.60, s, 1H; l: 7.73, d, 1H, Jₗⱼ=6.7 Hz; m: 7.90, s, 1H; n: 12.84 (exchangeable with D₂O), bs, 2H.

¹³C-NMR (DMSO-d₆, 8 ppm): C₁: 14.62, C_{I}: 21.65, C₂: 24.56, C₃: 32.09, C₄: 50.40, C_{II}: 69.80, C₅: 100.90, C₆: 105.18, C₇: 107.12, C₈: 108.92, C₉: 114.77, C₁₀: 119.46, C₁₁: 123.72, C₁₂: 126.17, C₁₃: 126.42, C₁₄: 126.83, C₁₅: 128.09, C₁₆: 129.34, C₁₇: 130.46, C₁₈: 132.92, C₁₉: 133.65, C₂₀: 133.97, C₂₁: 140.74, C₂₂: 145.52, C₂₃: 146.13, C₂₄: 146.53, C₂₅: 154.03, C₂₆: 164.91, C₂₇: 167.44, C₂₈: 175.00.

Anal.: Calc. for C₃₁H₂₉NO₈.½H₂O: C, 67.39; H, 5.52; N, 2.53. Found: C, 67.35; H, 5.21; N, 2.52.

### 6-(allyloxy)-3-(3-carboxybenzyl)-1-((6-ethylbenzo[d][1,3]dioxol-5-yl)methyl)-4-oxo-1,4-dihydroquinoline-2-carboxylic acid, 1r: (R═H₂C═CH—, R′═C₂H₅, R″═H):

Recrystallization from methanol-water (98:2) resulted in 0.21 g (80.1%) of pure product.

m.p.=165-166° C., TLC (70% EtOAc: 30% methanol) R_{f}=0.701.

IR: 3281 cm⁻¹ (carboxylic acid —OH), 1693 cm⁻¹ (a 1 unsaturated acid, C═O), 1693 cm⁻¹ (aromatic acid, C═O), 1590 cm⁻¹ (quinol-4-one, C═O).

¹H-NMR (DMSO-d₆, 8 ppm): a: 1.20, t, 3H, J_{ab}=7.5 Hz; b: 2.69, q, 2H, J_{ba}=7.4 Hz; c: 3.88, s, 2H; A: 4.62, d, 2H, J_{AD}=4.7 Hz; B: 5.26, d, 1H, J_{BD}=10.3 Hz; C, 5.39, d, 1H, J_{CD}=13.3 Hz; d: 5.41, s, 2H; e: 5.90, s, 1H; f: 5.94, s, 1H; D: 6.05, octet, 1H, J=5.5; g: 6.87, s, 1H; h: 7.31, d, 2H; i: 7.37, t, 1H, J=7.8 Hz; j: 7.54, d, 1H, Jⱼᵢ=7.4 Hz; k: 7.62, d, 1H; l: 7.73, d, 1H, Jₗⱼ=7.5 Hz; m: 7.89, s, 1H; n: 12.86 (exchangeable with D₂O), bs, 2H.

¹³C-NMR (DMSO-d₆, 8 ppm): C₁: 14.63, C₂: 24.56, C₃: 32.09, C₄: 50.43, C₁: 68.49, C₅: 100.91, C₆: 105.18, C₇: 106.47, C₈: 108.93, C₉: 114.87, C_{II}: 117.63, C₁₀: 119.42, C₁₁: 122.99, C₁₂: 126.17, C₁₃: 126.36, C₁₄: 126.84, C₁₅: 128.09, C₁₆: 129.33, C₁₇: 130.46, C₁₈: 132.93, C₁₉: 133.36, C₂₀: 133.95, C_{III}: 134.00, C₂₁: 140.74, C₂₂: 145.52, C₂₃: 146.22, C₂₄: 146.53, C₂₅: 154.75, C₂₆: 164.90, C₂₇: 167.45, C₂₈: 175.01.

Anal.: Calc. for C₃₁H₂₇NO₈. ½H₂O: C, 67.63; H, 5.09; N, 2.54. Found: C, 67.68; H, 5.01; N, 2.54.

### 3-(3-carboxybenzyl)-1-((6-ethylbenzo[d][1,3]dioxol-5-yl)methyl)-6-(methoxymethoxy)-4-oxo-1,4-dihydroquinoline-2-carboxylic acid, is (R═H₃C—O—CH₂—, R′═C₂H₅, R″═H)

Recrystallization from methanol-water (98:2) resulted in 0.21 g (66.7%) of pure product. m.p.=176-177° C., TLC (70% EtOAc: 30% methanol) R_{f}=0.610.

IR: 3443 cm⁻¹ (carboxylic acid —OH), 1698 cm⁻¹ (α, β unsaturated acid, C═O), 1698 cm⁻¹ (aromatic acid, C═O), 1587 cm⁻¹ (quinol-4-one, C═O).

¹H-NMR (DMSO-d₆, δ ppm): a: 1.21, t, 3H, J_{ab}=7.3 Hz; b: 2.71, q, 2H, J_{ba}=7.3 Hz; C, 3.38, s, 3H; c: 3.89, s, 2H; E: 5.26, s, 2H; d: 5.42, s, 2H; e: 5.90, s, 2H; f: 5.96, s, 1H; g: 6.88, s, 1H; h: 7.32, t, 1H, J=9.3 Hz; i: 7.37, d, 2H; j: 7.54, d, 1H, Jⱼᵢ=6.9 Hz; k: 7.74, d, 1H, Jₗⱼ=7.5 Hz; l: 7.79, s, 1H; m: 7.90, s, 1H; n: 12.82 (exchangeable with D₂O), bs, 2H.

¹³C-NMR (DMSO-d₆, δ ppm): C₁: 14.58, C₂: 24.54, C₃: 32.02, C₄: 50.42, C_{I}: 55.63, C_{III}: 94.07, C₅: 100.88, C₆: 105.16, C₇: 108.89, C₈: 109.08, C₉: 114.98, C₁₀: 119.31, C₁₁: 123.51, C₁₂: 126.08, C₁₃: 126.29, C₁₄: 126.82, C₁₅: 128.06, C₁₆: 129.26, C₁₇: 130.47, C₁₈: 132.86, C₁₉: 134.00, C₂₀: 134.52, C₂₁: 140.66, C₂₂: 145.52, C₂₃: 146.29, C₂₄: 146.54, C₂₅: 153.22, C₂₆: 164.80, C₂₇: 167.41, C₂₈: 175.09.

Anal.: Calc. for C₃₀H₂₇NO₈.H₂O: C, 63.94; H, 5.19; N, 2.49. Found: C, 63.87; H, 5.12; N, 2.62.

### 6-butoxy-3-(3-carboxybenzyl)-1-((6-ethylbenzo[d][1,3]dioxol-5-yl)methyl)-4-oxo-1,4-dihydroquinoline-2-carboxylic acid, 1t, (R=n-C₄H₉, R′═C₂H₅, R″═H)

Recrystallization from methanol-water (98:2) resulted in 0.21 g (77.7%) of pure product.

m.p.=161-162° C., TLC (70% EtOAc: 30% methanol) R_{f}=0.714.

IR: 3568 cm⁻¹ (carboxylic acid —OH), 1686 cm⁻¹ (α β unsaturated acid, C═O), 1686 cm⁻¹ (aromatic acid, C═O), 1589 cm⁻¹ (quinol-4-one, C═O).

¹H-NMR (DMSO-d₆, δ ppm): A: 0.93, t, 3H, J_{AB}=7.4 Hz; a: 1.21, t, 3H, J_{ab}=7.5 Hz; B: 1.43, sextet, 2H, J=7.5 Hz; C, 1.72, quintet, 2H, J=6.9 Hz; b: 2.69, q, 2H, J_{ba}=7.5 Hz; c: 3.89, s, 2H; D: 4.05, t, 2H, J_{DC}=6.5 Hz; d: 5.42, s, 2H; e: 5.89, s, 1H; f: 5.93, s, 1H; g: 6.87, s, 1H; h: 7.28, d, 2H; i: 7.36, t, 1H, J=7.7 Hz; j: 7.54, d, 1H, Jⱼᵢ=7.7 Hz; k: 7.62, s, 1H; l: 7.73, d, 1H, Jₗⱼ=7.7 Hz; m: 7.91, s, 1H; n: 12.84 (exchangeable with D₂O), bs, 2H.

¹³C-NMR (DMSO-d₆, δ ppm): C_{I}: 13.58, C₁: 14.55, C_{II}: 18.64, C₂: 24.51, C_{III}: 30.55, C₃: 32.02, C₄: 50.37, C_{IV}: 67.52, C₅: 100.85, C₆: 105.14, C₇: 105.92, C₈: 108.87, C₉: 114.85, C₁₀: 119.31, C₁₁: 122.93, C₁₂: 126.13, C₁₃: 126.37, C₁₄: 126.78, C₁₅: 128.03, C₁₆: 129.28, C₁₇: 130.45, C₁₈: 132.85, C₁₉: 133.79, C₂₀: 133.96, C₂₁: 140.70, C₂₂: 145.49, C₂₃: 146.02, C₂₄: 146.50, C₂₅: 155.25, C₂₆: 164.83, C₂₇: 167.39, C₂₈: 175.99.

Anal.: Calc. for C₃₂H₃₁NO₈.H₂O: C, 66.77; H, 5.78; N, 2.43. Found: C, 67.14; H, 5.77; N, 2.48.

### 3-(3-carboxybenzyl)-1-((6-ethylbenzo[d][1,3]dioxol-5-yl)methyl)-6-isobutoxy-4-oxo-1,4-dihydroquinolin-2-carboxylic, 1u (R=i-C₄H₉, R′═C₂H₅, R″═H)

Recrystallization from methanol-water (98:2) resulted in 0.22 g (81.4%) of pure product.

m.p.=185-186° C., TLC (70% EtOAc: 30% methanol) R_{f}=0.727.

IR: 3456 cm⁻¹ (carboxylic acid —OH), 1699 cm⁻¹ (α β unsaturated acid, C═O), 1699 cm⁻¹ (aromatic acid, C═O), 1584 cm⁻¹ (quinol-4-one, C═O).

¹H-NMR (DMSO-d₆, δ ppm): A: 0.98, d, 6H, J_{AB}=6.7 Hz; a: 1.20, t, 3H, J_{ab}=7.4 Hz; B: 2.03, nonet, 1H, J=6.5 Hz; b: 2.69, q, 2H, J_{ba}=7.4 Hz; C, 3.82, d, 2H, J=6.4 Hz; c: 3.89, s, 2H; d: 5.41, s, 2H; e: 5.89, s, 1H; f: 5.91, s, 1H; g: 6.87, s, 1H; h: 7.31, d, 2H; i: 7.36, t, 1H, J=7.7 Hz; j: 7.54, d, 1H, Jⱼᵢ=7.6 Hz; k: 7.62, s, 1H; l: 7.73, d, 1H, Jₗⱼ=7.6 Hz; m: 7.91, s, 1H; n: 12.84 (exchangeable with D₂O), bs, 2H.

¹³C-NMR (DMSO-d₆, δ ppm): C₁: 14.63, C_{I}: 18.98, C₂: 24.56, C_{II}: 27.59, C₃: 32.07, C₄: 50.41, C_{III}: 74.06, C₅: 100.91, C₆: 105.16, C₇: 105.94, C₈: 108.93, C₉: 114.86, C₁₀: 119.39, C₁₁: 123.01, C₁₂: 126.18, C₁₃: 126.39, C₁₄: 126.84, C₁₅: 128.09, C₁₆: 129.33, C₁₇: 130.47, C₁₈: 132.93, C₁₉: 133.81, C₂₀: 133.99, C₂₁: 140.75, C₂₂: 145.52, C₂₃: 146.11, C₂₄: 146.53, C₂₅: 155.36, C₂₆: 164.91, C₂₇: 167.46, C₂₈: 175.03.

Anal.: Calc. for C₃₂H₃₁NO₈.H₂O: C, 66.77; H, 5.78; N, 2.43. Found: C, 66.73; H, 5.77; N, 2.49.

### Ethyl 6-sec-butoxy-3-(3-(ethoxycarbonyl)benzyl)-1-((6-ethylbenzo[d][1,3]dioxol-5-yl)methyl)-4-oxo-1,4-dihydroquinoline-2-carboxylate, 1v (R=sec-C₄H₉, R′═C₂H₅, R″═H)

Recrystallization from methanol-water (98:2) resulted in 0.16 g (67.4%) of pure product.

m.p.=229-231° C., TLC (70% EtOAc: 30% methanol) R_{f}=0.727.

IR: 3456 cm⁻¹ (carboxylic acid —OH), 1699 cm⁻¹ (α, β unsaturated acid, C═O), 1699 cm⁻¹ (aromatic acid, C═O), 1588 cm⁻¹ (quinol-4-one, C═O).

¹H-NMR (DMSO-d₆, δ ppm): A: 0.91, t, 3H, J_{AB}=7.1 Hz; a: 1.20, t, 3H, J_{ab}=7.2 Hz; B: 1.25, d, 3H, J=5.3 Hz; C, 1.61, octet, 1H, J=6.5 Hz; D: 1.66, octet, 1H, J=6.4 Hz; b: 2.69, q, 2H, J_{ba}=7.1 Hz; c: 3.87, s, 2H; E: 4.44, sextet, 1H, J=5.7 Hz; d: 5.40, s, 2H; e: 5.89, s, 2H; f: 5.95, s, 1H; g: 6.87, s, 1H; h: 7.27, d, 2H; i: 7.36, t, 1H, J=7.5 Hz; j: 7.54, d, 1H, Jⱼᵢ=6.9 Hz; k: 7.59, s, 1H; l: 7.73, d, 1H, Jₗⱼ=6.9 Hz; m: 7.90, s, 1H; n: 12.83 (exchangeable with D₂O), bs, 2H.

¹³C-NMR (DMSO-d₆, δ ppm): C_{I}: 9.50, C₁: 14.61, C_{II}: 18.86, C₂: 24.55, C_{III}: 28.43, C₃: 32.10, C₄: 50.40, C_{IV}: 74.76, C₅: 100.89, C₆: 105.18, C₇: 107.15, C₈: 108.91, C₉: 114.75, C₁₀: 119.47, C₁₁: 123.70, C₁₂: 126.16, C₁₃: 126.41, C₁₄: 126.82, C₁₅: 128.08, C₁₆: 129.33, C₁₇: 130.45, C₁₈: 132.91, C₁₉: 133.64, C₂₀: 133.96, C₂₁: 140.74, C₂₂: 145.51, C₂₃: 146.14, C₂₄: 146.52, C₂₅: 154.36, C₂₆: 164.89, C₂₇: 167.43, C₂₈: 174.98.

Anal.: Calc. for C₃₂H₃₁NO₈: C, 68.73; H, 5.60; N, 2.51. Found: C, 68.34; H, 5.70; N, 2.50.

### 3-(3-carboxybenzyl)-6-(cyclopropylmethoxy)-1-((6-ethylbenzo[d][1,3]dioxol-5-yl)methyl)-4-oxo-1,4-dihydroquinoline-2-carboxylic acid, 1w (R=cyclo-C₃H₅—CH₂—, R′═C₂H₅, R″═H)

Recrystallization from methanol-water (98:2) resulted in 0.16 g (67.4%) of pure product.

m.p.=229-231° C., TLC (70% EtOAc: 30% methanol) R_{f}=0.727.

IR: 3446 cm⁻¹ (carboxylic acid —OH), 1692 cm⁻¹ (α, β unsaturated acid, C═O), 1692 cm⁻¹ (aromatic acid, C═O), 1589 cm⁻¹ (quinol-4-one, C═O).

¹H-NMR (DMSO-d₆, δ ppm): A: 0.33, d, 2H, J_{AB}=4.4 Hz; A: 0.56, d, 2H, J_{BA}=7.8 Hz; a: 1.20, t, 3H, J_{ab}=7.4 Hz; C, 1.20, t, 1H, J=7.4 Hz; b: 2.69, q, 2H, J_{ba}=7.1 Hz; c: 3.87, s, 2H; D: 3.89, d, 2H, J=6.4 Hz; d: 5.41, s, 2H; e: 5.89, s, 2H; f: 5.95, s, 1H; g: 6.87, s, 1H; h: 7.30, d, 2H; i: 7.36, t, 1H, J=7.5 Hz; j: 7.53, d, 1H, Jⱼᵢ=7.5 Hz; k: 7.58, s, 1H; l: 7.73, d, 1H, Jₗⱼ=7.6 Hz; m: 7.89, s, 1H; n: 12.84 (exchangeable with D₂O), bs, 2H.

¹³C-NMR (DMSO-d₆, δ ppm): C_{I}: 3.12, C_{II}: 10.00, C₁: 14.61, C₂: 24.55, C₃: 32.04, C₄: 50.38, C_{III}: 72.40, C₅: 100.89, C₆: 105.14, C₇: 105.84, C₈: 108.92, C₉: 114.85, C₁₀: 119.36, C₁₁: 123.08, C₁₂: 126.16, C₁₃: 126.37, C₁₄: 126.83, C₁₅: 128.08, C₁₆: 129.30, C₁₇: 130.45, C₁₈: 132.91, C₁₉: 133.77, C₂₀: 133.99, C₂₁: 140.73, C₂₂: 145.50, C₂₃: 146.03, C₂₄: 146.52, C₂₅: 154.23, C₂₆: 164.88, C₂₇: 167.44, C₂₈: 174.99.

Anal.: Calc. for C₃₂H₂₉NO₈. ½H₂O: C, 68.08; H, 5.31; N, 2.48. Found: C, 68.18; H, 5.31; N, 2.46.

### 3-(3-carboxybenzyl)-1-((6-ethylbenzo[d][1,3]dioxol-5-yl)methyl)-4-oxo-6-(pentyloxy)-1,4-dihydroquinoline-2-carboxylic acid, 1x (R=n-C₅H₁₁, R′═C₂H₅, R″═H)

Recrystallization from methanol-water (98:2) resulted in 0.21 g (66.7%) of pure product.

m.p.=152-155° C., TLC (70% EtOAc: 30% methanol) R_{f}=0.766.

IR: 3568 cm⁻¹ (carboxylic acid —OH), 1684 cm⁻¹ (α, β unsaturated acid, C═O), 1684 cm⁻¹ (aromatic acid, C═O), 1589 cm⁻¹ (quinol-4-one, C═O).

¹H-NMR (DMSO-d₆, δ ppm): A: 0.89, t, 3H, J_{AB}=4.7 Hz; a: 1.20, t, 3H, J_{ab}=7.4 Hz; B: 1.33-1.37, m, 4H, J=7.7 Hz; C, 1.73, quintet, 2H, J=6.9 Hz; b: 2.69, q, 2H, J_{ba}=7.3 Hz; c: 3.88, s, 2H; D: 4.03, t, 2H, J_{DC}=5.9 Hz; d: 5.41, s, 2H; e: 5.89, s, 2H; f: 5.91, s, 1H; g: 6.87, s, 1H; h: 7.29, d, 2H; i: 7.37, t, 1H, J=8.4 Hz; j: 7.54, d, 1H, Jⱼᵢ=7.6 Hz; k: 7.62, s, 1H; l: 7.73, d, 1H, Jₗⱼ=6.5 Hz; m: 7.89, s, 1H; n: 12.83 (exchangeable with D₂O), bs, 2H.

¹³C-NMR (DMSO-d₆, δ ppm): C_{I}: 13.86, C₁: 14.59, C_{II}: 21.82, C₂: 24.53, C_{II}: 27.63, C_{III}: 28.21, C₃: 32.02, C₄: 50.37, C_{IV}: 67.52, C₅: 100.88, C₆: 105.16, C₇: 106.09, C₈: 108.89, C₉: 114.81, C₁₀: 119.34, C₁₁: 122.95, C₁₂: 126.17, C₁₃: 126.37, C₁₄: 126.79, C₁₅: 128.05, C₁₆: 129.30, C₁₇: 130.59, C₁₈: 132.89, C₁₉: 133.97, C₂₀: 134.87, C₂₁: 140.74, C₂₂: 145.49, C₂₃: 146.50, C₂₄: 146.71, C₂₅: 155.25, C₂₆: 164.86, C₂₇: 167.42, C₂₈: 174.99.

Anal.: Calc. for C₃₃H₃₃NO₈. ½H₂O: C, 68.27; H, 5.68; N, 2.41. Found: C, 68.04; H, 5.77; N, 2.48.

### 3-(3-carboxybenzyl)-1-((6-ethylbenzo[d][1,3]dioxol-5-yl)methyl)-6-(3-hydroxypropoxy)-4-oxo-1,4-dihydroquinoline-2-carboxylic acid, 1y (R═HO—H₂C—CH₂—CH₂—, R′═C₂H₅, R″═H)

Recrystallization from methanol-water (98:2) resulted in 0.14 g (74.0%) of pure product.

m.p.=192-194° C., TLC (70% EtOAc: 30% methanol) R_{f}=0.545.

IR: 3392 cm⁻¹ (carboxylic acid —OH), 1690 cm⁻¹ (α, β unsaturated acid, C═O), 1690 cm⁻¹ (aromatic acid, C═O), 1586 cm⁻¹ (quinol-4-one, C═O).

¹H-NMR (DMSO-d₆, δ ppm): a: 1.20, t, 3H, J_{ab}=7.5 Hz; A: 1.88, quintet, 2H, J=6.1 Hz; b: 2.69, q, 2H, J_{ba}=7.4 Hz; B: 3.55, t, 2H, J_{BA}=7.4 Hz; c: 3.88, s, 2H; C, 4.11, t, 2H, J_{CA}=6.1 Hz; d: 5.41, s, 2H; e: 5.89, s, 2H; f: 5.91, s, 1H; g: 6.87, s, 1H; h: 7.29, d, 2H; i: 7.36, t, 1H, J=7.5 Hz; j: 7.54, d, 1H, Jⱼᵢ=7.6 Hz; k: 7.60, s, 1H; l: 7.73, d, 1H, Jₗⱼ=7.6 Hz; m: 7.89, s, 1H; n: 12.84 (exchangeable with D₂O), bs, 2H.

¹³C-NMR (DMSO-d₆, δ ppm): C₁: 14.59, C₂: 24.53, C_{3 & I}: 31.97, C₄: 50.39, C_{II}: 57.24, C_{III}: 65.03, C₅: 100.88, C₆: 105.14, C₇: 105.90, C₈: 108.90, C₉: 114.85, C₁₀: 119.36, C₁₁: 122.98, C₁₂: 126.14, C₁₃: 126.37, C₁₄: 126.82, C₁₅: 128.08, C₁₆: 129.28, C₁₇: 130.46, C₁₈: 132.88, C₁₉: 133.79, C₂₀: 133.98, C₂₁: 140.71, C₂₂: 145.49, C₂₃: 146.02, C₂₄: 146.51, C₂₅: 155.28, C₂₆: 164.85, C₂₇: 167.42, C₂₈: 175.01.

Anal.: Calc. for C₃₁H₂₉NO₉. ½H₂O: C, 65.49; H, 5.10; N, 2.46. Found: C, 65.24; H, 5.27; N, 2.59.

In vitro binding assay. Varying concentrations of HJP272, ranging from 10³ to 10⁸ pM, were incubated with 10 ug of BioXTal ET-A or ET-B receptor and 224 pM [¹²⁵I]Tyr13-ET-1 in 50 mM NaH₂PO₄, 150 mM NaCl, 5 mM EDTA, and 0.1% bovine serum albumin.

For total binding, radio labeled ET-1 and receptor were incubated without the receptor antagonist. For non-specific binding, 300 nM cold ET-1 was substituted for the antagonist. Assay mixtures were incubated at 30 to 32° C. for two hours and the assays were stopped by the addition of cold buffer. Assay mixtures were then quickly filtered through GF/C Whatman filters and washed with cold assay buffer in a Millipore sampling manifold. Filters were collected and counts per minute of the bound radio labeled ET-1 were read by a Packard Cobra Gamma Counter (Perkin Elmer).

Percent specific binding was calculated for all data points. Assays containing ET-A receptor were run in triplicate and assays containing the ET-B receptor were run in duplicate.

Animal models of preterm birth. The Animal Care and Utilization Committee of the College of Pharmacy and Allied Health Professions, St. John's University approved all experimental protocols, and the research was conducted according to the requirements of NIH Guide for the Care and Use of Laboratory Animals (revised 1996). C57B1/6 mice from Taconic Laboratory were used for all experiments. Animals were housed in plastic cages in a temperature-controlled animal facility with alternating 12:12 hour light-dark cycles, with ad libitum access to food and water.

Nineteen timed pregnant C57B1/6 E15.5 mice were injected with LPS in 0.5 ml PBS at a concentration of 50 mg/kg. These mice were randomly assigned to two groups. Group A mice (n=8) received 50 mg/kg HJP272 dissolved in PBS and Group B mice (n=11) received PBS only.

All treatments were administered by ip injection 10 hours after LPS injection. Mice were continually monitored and the time of delivery of all pups, as well as the number of pups delivered, was recorded. All mice were autopsied at the end of the experiment to confirm pregnancy and to determine the number of pups retained in utero.

Animal models of smoking related acute lung injury. The Animal Care and Utilization Committee of the College of Pharmacy and Allied Health Professions, St. John's University approved all experimental protocols, and the research was conducted according to the requirements of NIH Guide for the Care and Use of Laboratory Animals (revised 1996).

Female Syrian hamsters, weighing approximately 100 g each, were divided into two groups as follows: Group 1 was treated IP with: 1) 500 μg of HJP272 in 0.5 mL Na₂CO₃ and 2) 5 μg ET-1 in 0.25 mL PBS 60 minutes later, followed immediately by exposure to cigarette smoke for two hours. This regimen was continued for three days.

Group 2 was treated IP with: 1) 0.5 mL Na₂CO₃ and 2) μg ET-1 in 0.25 mL PBS 60 minutes later, followed immediately by exposure to cigarette smoke for three consecutive days as per Group 1. The animals were placed in a 70×48×38 cm chamber and passively exposed to cigarette smoke produced by a TE-10 smoking machine (Teague Enterprises, Davis, Calif.) that simultaneously burned two filtered research-grade cigarettes (type 2R4F, University of Kentucky). Smoke from both the filtered and burning ends of the cigarette was collected, and then mixed together with air in a separate chamber, thus simulating second-hand smoke exposure. Each cigarette was puffed once per minute for two seconds at a flow rate of 1.05 LPM, yielding 35 mL of smoke. This cycle was repeated nine times before ejecting the cigarette and loading a new one. Proper flow rate was maintained by a vacuum pump that established negative pressure at the exhaust port. Total smoke particulates averaged 87 mg per cubic meter.

All animals were sacrificed 24 hours after completing the smoke exposure regimen to maximize the neutrophil population in the lung, and the percentage of BALF neutrophils was measured (van der Vaart et al. 2004)

### Results

In vitro binding assay. The competitive binding assay with radio labeled ligand shows binding of HJP272 to the endothelin A receptor with an IC₅₀ of 70.1 nM (FIG. 2).

Furthermore, the compound is a selective antagonist, with the ratio of IC₅₀ for the endothelin A receptor to IC₅₀ for the endothelin B receptor of 219.2.

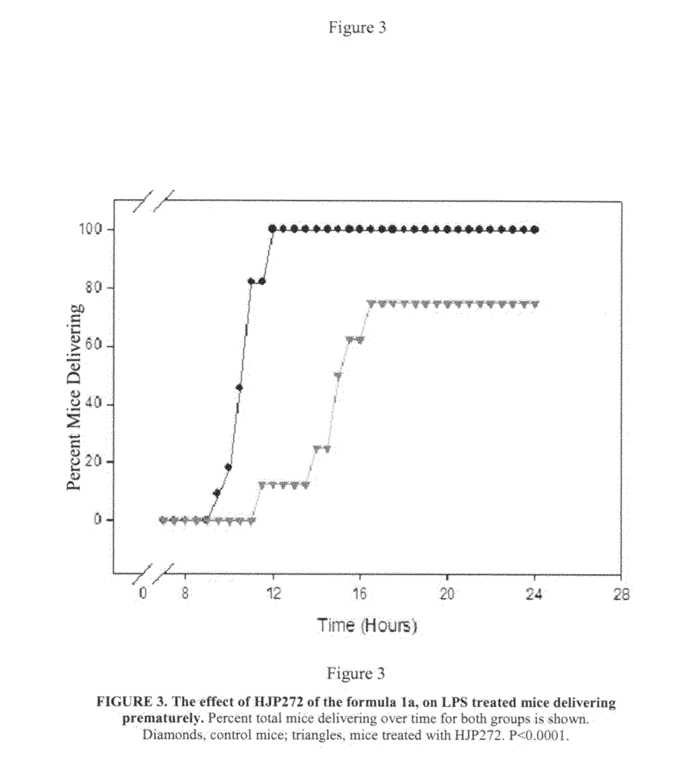

Animal models of preterm birth. Treatment with HJP272 of formula 1a controlled preterm labor and delivery in our mouse model of infection-associated preterm labor very effectively (Olgun et al. 2008). Among mice treated at E15.5 with a high dose (50 mg/kg) of LPS, six out of eight mice treated with HJP272 delivered prematurely as compared to 11 out of 11 of the control mice treated with only PBS. (FIG. 3).

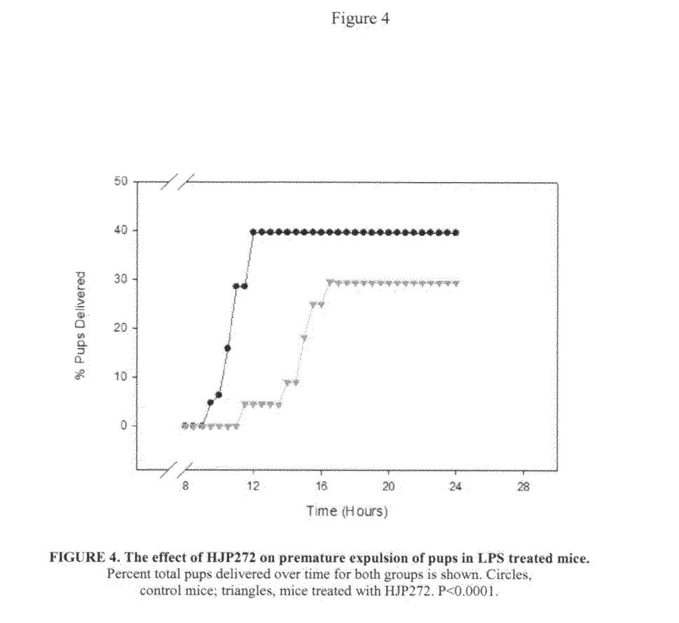

The percent of prematurely delivered pups in the group of mice treated with the ET_{A}-RA was 30.0 (13 out of 44) and the percent of prematurely delivered pups in the control group treated with PBS only was 39.7 (25 out of 63) (FIG. 4).

The average delivery time of the six HJP272-treated mice that delivered, however was T15.0+/−1.8 hours, while the average delivery time of the control mice treated with only PBS was T10.9+/−0.61 hours. This shift in time of delivery, in the setting of the high dose of LPS, was statistically significant (p<0.0001).

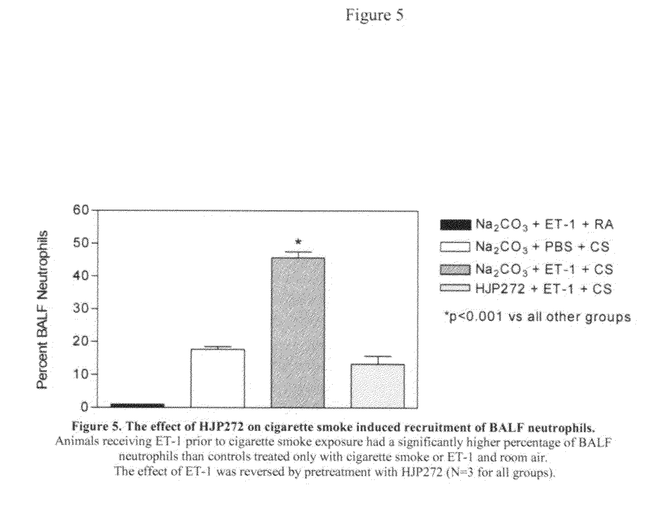

Animal models of smoking related acute lung injury. Treatment with ET-1 prior to exposure to cigarette smoke significantly increased the percentage of BALF neutrophils in hamsters (Bhavsar et al. 2008b). As shown in FIG. 5, hamsters receiving ET-1 had 46% neutrophils compared to 18% and 1% for controls treated only with cigarette smoke or ET-1 and room air, respectively. Furthermore, pretreatment with the ET_{A}-RA reversed this effect (FIG. 5).

### Discussion

In this work, we have shown that 3-(3-carboxybenzyl)-1-((6-ethylbenzo[d][1,3]dioxol-5-yl)methyl)-6-hydroxy-4-oxo-1,4-dihydroquinoline-2-carboxylic acid (HJP272), a novel 1,3,6-trisubstituted-2-carboxy-quinol-4-one and

ET_{A}-RA, is very effective in controlling both preterm delivery and acute lung inflammation in animal models. While endothelin has now been implicated in a very broad spectrum of disease processes, ranging from cancer to cardiovascular disease, its role in the pathogenesis of reproductive disorders, such as premature delivery, has not been thoroughly investigated.

The enthusiasm for endothelin as a therapeutic target in reproductive disorders is dampened by our understanding, from work with both ET-1 and ECE-1 knockout mice (Yanagisawa et al. 1998), that blockade of ET-1 action early in gestation leads to severe craniofacial anomalies and death in the perinatal period. None of the pups in the work presented here demonstrated such abnormalities, probably because exposure to the ET-1 blocking agent was transient and relatively late in gestation, well after embryogenesis and organogenesis had been completed.

The results of the testing of animal models of cigarette smoking related acute lung injury are consistent with selective recruitment of neutrophils by ET-1, and suggest that this mediator may play an important role in modulating the pulmonary inflammatory response. The level of ET-1 synthesis could determine whether the inflammatory cell population is predominantly composed of neutrophils or monocytes.

Such variation in the nature of the inflammatory response could have important implications for the type and extent of lung damage resulting from exposure to different pulmonary toxins.

While the mechanism responsible for the specific effect of ET-1 on neutrophils remains unclear, it may involve upregulation of ICAM-1, which facilitates their adhesion to vascular endothelium (Zhang et al. 2006). Alternatively, ET-1 could increase neutrophil expression of CXCR2, a cell-surface receptor that binds interleukin-8, a potent activator of these cells (DiVietro et al. 2001; Reutershan et al. 2006). Either of these processes could be enhanced by the fact that ET-1 can change the F-actin content of neutrophils, thereby promoting their sequestration in pulmonary microvessels (Sato et al. 2000).

In the current studies, treatment with the ET_{A}-RA reversed the effect of ET-1 on neutrophil recruitment to the lung. This finding may reflect an impaired ability of these cells to adhere to pulmonary vascular endothelium. Further support for this concept is found in studies of inflammatory bowel disease, where treatment with the nonselective endothelin receptor antagonist, bosentan, resulted in markedly reduced attachment of neutrophils to colonic submucosal venules (Anthoni et al. 2006).

With regard to the cellular composition of inflammatory reactions, there is increasing evidence that ET-1 confers a selective advantage on neutrophils, allowing them to populate the lung at the expense of other inflammatory cells. Such a process might have important implications for the treatment of inflammatory lung disorders, including COPD. Reducing ET-1 activity with endothelin receptor antagonists could potentially decrease neutrophil-derived enzymes and oxidants in the lung, thereby slowing the progression of lung injury.

The data presented in the current work demonstrate the potential use of the ET_{A}-RA for two extremely important clinical problems, namely preterm birth and acute lung injury. No satisfactory therapy currently exists for these disorders. Furthermore, because ET-1 is implicated in such a broad range of pathological processes, the synthetic ET_{A}-RAs may have clinical value in any one of a number of disorders, including ones that have extremely broad impact, such as cardiovascular disease and neoplasia.

### REFERENCES

Anthoni A M, Revert F, Moya A et al. 2000. Intravenous B Q-123 and phosphoramidon reduce ventricular ectopic beats and myocardial infarct size in dogs submitted to coronary occlusion and reperfusion. Gen Pharmacol 35: 143-147.
Bhavsar T M, Cerreta J M, Liu M, Reznik S E and Cantor J O. 2008. Phosphoramidon, an endothelin-converting enzyme inhibitor, attenuates lipopolysaccharide-induced acute lung injury. Exper Lung Res 34: 141-154. (a)
Bhavsar T M, Liu X J, Patel H Stephani R and Cantor J O. 2008. Preferential recruitment of neutrophils by endothelin-1 in acute lung inflammation induced by lipopolysaccharide or cigarette smoke. Internat J COPD, in press. (b)
DiVietro J A, Smith M J, Smith B R E et al. 2001. Immobilized I L-8 triggers progressive activation of neutrophils rolling in vitro on p-selectin and intercellular adhesion molecule-1. J Immunol 167: 4017-4025.
Kaya T, Cetin A, Cetin M and Sarioglu Y. 1999. Effects of endothelin-1 and calcium channel blockers on contractions in human myometrium. A study on myometrial strips from normal and diabetic pregnant women. J Reprod Med 44: 155-121.
Koscica K, Sylvestre G and Reznik S E. 2004. The effect of phosphoramidon on inflammation-mediated preterm delivery in a mouse model. Am J Obstet Gynecol 190: 528-531.
Olgun N, Patel H, Wang W, Yen H, Stephani R and Reznik S E. 2008. The Role of a 1,3,6-trisubstituted-2-carboxy-quinol-4-one, a novel putative selective ET_{A} antagonist, in controlling preterm labor in a mouse model. Can J Physiol Pharmacol 86: 571-575.
Reutershan J, Morris M A, Burcin T L et al. 2006. Critical role of endothelial CXCR2 in LPS-induced neutrophil migration into the lung. J Clin Invest 116: 695-702.
Sato Y, Hogg J C, English D et al. 2000. Endothelin-1 changes polymorphonuclear leukocytes' deformability and CD11b expression and promotes their retention in the lung. Amer J. Respir Cell Mol Biol 23: 7-10.
Van der Vaart H, Postma D S, Timens W et al. 2004. Acute effects of cigarette smoke on inflammation and oxidative stress: a review. Thorax 59: 713-721.
Wang W, Yen H, Chen C-H, Soni R, Jasani N, Sylvestre G and Reznik S E. 2008. The Endothelin Converting Enzyme-1 (ECE-1)/Endothelin-1 (ET-1) Pathway Plays a Critical Role in Infection-Associated Premature Delivery in a Mouse Model. Am J Path, in press.
Wolff K, Nisell H, Modin A, Lundberg J M, Lunell N O and Lindblom B. 1993. Contractile effects of endothelin 1 and endothelin 3 on myometrium and small intramyometrial arteries of pregnant women at term. Gynecol Obstet Invest 36: 166-171.
Woods M, Mitchell J A, Wood E G, Barker S, Walcont N R, Rees G M & Warn T D. 1999. Endothelin-1 is induced by cytokines in human vascular smooth muscle cells: evidence for intracellular endothelin-converting enzyme. Molec Pharm 55: 902-909.
Yallampalli C and Garfield R E. 1994. Uterine contractile responses to endothelin-1 and endothelin receptors are elevated during labor. Biol Reprod 51: 640-645.
Yanagisawa M, Kurihara H, Kimura S, Tomobe Y, Kobayishi Y, Mitsui Y, Yazaki Y, Goto K and Masaki T. 1988. A novel potent vasoconstrictor peptide produced by vascular endothelial cells. Nature 332: 411-415.
Yanagisawa H, Yanagisawa M, Kapur R P, Richardson J A, Williams S C, Cloutheir D E, de Wit D, Noriaki E and Hammer R E. 1998. Dual genetic pathways of endothelin mediated intercellular signaling revealed by targeted disruption of endothelin converting enzyme-1 gene. Development 125: 825-836.
Zhang J S, Tan Y R, Xiang Y et al. 2006. Regulatory peptides modulate adhesion of polymorphonuclear leukocytes to bronchial epithelial cells through regulation of interleukins, ICAM-1 and NF-kappa B/Ikappa B. Acta Biochim Biophys Sin 38: 119-128.

What is claimed is:

1. A method of treating disease in a mammal comprising the administration of pharmaceutically effective amount of any one or combination of two or more compounds consisting of a novel series of 1,3,6-trisubstituted-2-carboxy-quinol-4-ones and pharmaceutically acceptable carrier
A method of antagonizing the action of endothelin-1, consisting of administering to a human a pharmaceutically effective amount of any one or combination of two or more of a novel series of 1,3,6-trisubstituted-2-carboxy-quinol-4-ones and pharmaceutically acceptable carrier
A method of claim 1, wherein the said compound or compounds are administered orally, intravenously, topically, intramuscularly, or by any other route deemed efficacious
A method of claim 1, wherein the mammal is human
A method of claim 1 wherein the pharmaceutical carrier is saline solution, DMSO, an alcohol, sodium carbonate solution, or water.
A method of claim 1, wherein the effective dose is from about 1 □g/kg to 10 mg/kg of body weight
A method of claim 1 wherein the disease to be treated is selected from one or more of the following disorders that may involve disturbances in endothelin-1 production, and are therefore suitable for treatment with one or more of the said compounds: hypertension; heart failure; arterial injury; reperfusion injury; angina; acute or chronic pulmonary hypertension; cerebral ischemia; myocardial ischemia; cerebral vasospasm; atherosclerosis; emphysema; asthma; bronchitis; bronchiectasis; pneumonia; adult respiratory syndromes; neonatal respiratory distress syndrome; bronchopulmonary dysplasia; interstitial fibrosis; cystic fibrosis; persistent pulmonary hypertension of the newborn; proliferative diseases and neoplasia, especially prostate cancer; acute and chronic renal failure; cyclosporin-induced nephrotoxicity; gastric ulceration; colitis; migraine; Raynaud's disease; erectile dysfunction; endotoxin-induced toxicity; LPL-related lipoprotein disorders; platelet disorders; thrombosis; IL-2 mediated cardiotoxicity; nociception; preterm labor; premature rupture of membranes; placental abruption; pre-eclampsia/eclampsia; stillbirth; miscarriage and cancer-related bone pain.
